(19) United States
(12) Patent Application Publication
(10) Pub. No.: US 2017/0014524 A1
(43) Pub. Date: Jan. 19, 2017

(54) AURISTATIN-ANTIBODY CONJUGATES AND USES THEREOF

(71) Applicant: The California Institute for Biomedical Research, La Jolla, CA (US)

(72) Inventors: Arnab K. CHATTERJEE, San Diego, CA (US); Feng WANG, Carlsbad, CA (US); Stephanie PINKERTON, San Diego, CA (US); Peter G. SCHULTZ, La Jolla, CA (US)

(21) Appl. No.: 15/210,729

(22) Filed: Jul. 14, 2016

Related U.S. Application Data
(60) Provisional application No. 62/192,768, filed on Jul. 15, 2015.

(51) Int. Cl. A61K 47/48; C07K 16/28

(52) U.S. Cl. CPC A61K 47/48415; C07K 16/2866; A61K 47/48569; C07K 2317/92

(57) ABSTRACT

Disclosed herein are auristatin-antibody conjugates and compositions thereof. The auristatin-antibody conjugates and compositions may be used for treating diseases such as cancer. Also disclosed are methods of producing auristatin-antibody conjugates.

### CROSS-REFERENCE

This application claims the benefit of U.S. Provisional Application No. 62/192,768, filed Jul. 15, 2015, which application is incorporated herein by reference.

### SEQUENCE LISTING

The instant application contains a Sequence Listing which has been submitted electronically in ASCII format and is hereby incorporated by reference in its entirety. Said ASCII copy, created on Jul. 6, 2016, is named 41135-749_201_SL.txt and is 9,639 bytes in size.

### BACKGROUND OF THE INVENTION

Monomethyl auristatins are highly potent antimitotic and antineoplastic agents used to treat Hodgkin's lymphoma, as well as other types of cancer. There are currently three types of monomethyl auristatin: monomethyl auristatin E (MMAE), monomethyl auristatin F, (MMAF), and monomethyl auristatin D (MMAD). Because of their toxicities, auristatins may be delivered in the form of an antibody drug conjugate, where the antibody portion serves to target the potent drugs to cancer cells. Recently, such antibody drug conjugates have been developed using anti-CD3 (e.g., brentuximab) and anti-GPNMB (e.g., glembatumumab).

### SUMMARY OF THE INVENTION

In one aspect, provided herein are antibody drug conjugates comprising an auristatin linked to an antibody or fragment thereof through a phenyl group of the auristatin.

The antibody drug conjugate may comprise an auristatin of Formula (I):

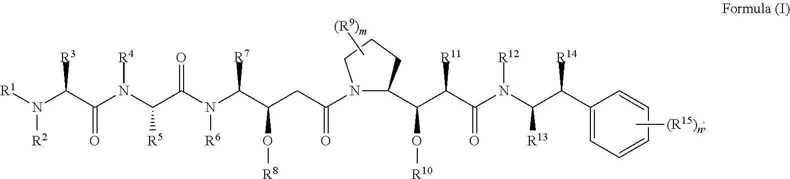

wherein
each R¹, R², R⁴, R⁶, and R¹² is independently hydrogen or optionally substituted alkyl;
each R³, R⁵, R⁷, and R¹¹ is independently optionally substituted alkyl;
each R⁸ and R¹⁰ is independently optionally substituted alkyl;
each R¹³ and R¹⁴ are independently hydrogen, halogen, —CN, —OR^{a}, —COOR^{a}, —N(R^{a})₂, optionally substituted alkyl, optionally substituted C₃-C₈ cycloalkyl, optionally substituted C₂-C₈ heterocycloalkyl, optionally substituted aryl, or optionally substituted 5-, or 6-membered heteroaryl;
each R⁹ is independently hydrogen, halogen, —OR^{a}, —N(R^{a})₂, —SO₂N(R^{a})₂, —CO₂N(R^{a})₂, or optionally substituted alkyl; or 2 R⁹ on the same carbon are taken together to form an oxo; or 2 R⁹ on the adjacent carbons are taken together to form an alkene;
each R¹⁵ is independently hydrogen, halogen, —OR^{a}, —N(R^{a})₂, —SO₂N(R^{a})₂, —CO₂N(R^{a})₂, or optionally substituted alkyl;
each R^{a} is hydrogen or optionally substituted alkyl;
n is 0-5; and
m is 0-3.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; and n is 0.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is methyl; R¹³ is methyl; and R¹⁴ is —OH.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is hydrogen; R¹³ is methyl; and R¹⁴ is —OH.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is methyl; R¹³ is —COOH; and R¹⁴ is hydrogen.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is hydrogen; R¹³ is —COOH; and R¹⁴ is hydrogen.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is methyl; R¹³ is 2-thiazolyl; and R¹⁴ is hydrogen.

The antibody drug conjugate may comprise an auristatin of Formula (I), wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is hydrogen; R¹³ is 2-thiazolyl; and R¹⁴ is hydrogen.

The antibody drug conjugate may comprise an auristatin of Formula (II), wherein the antibody or fragment thereof is linked through a primary amine on a phenyl group of the auristatin amine of Formula (II):

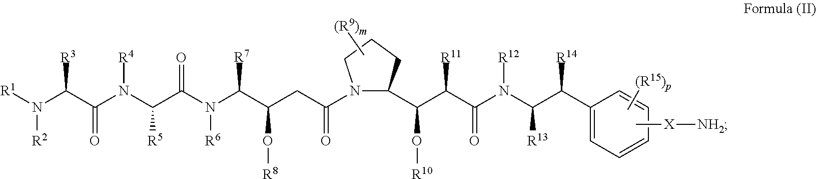

wherein
each R¹, R², R⁴, R⁶, and R¹² is independently hydrogen or optionally substituted alkyl;
each R³, R⁵, R⁷, and R₁₁ is independently optionally substituted alkyl;
each R⁸ and R¹⁰ is independently optionally substituted alkyl;
each R¹³ and R¹⁴ are independently hydrogen, halogen, —CN, —OR^{a}, —COOR^{a}, —N(R^{a})₂, optionally substituted alkyl, optionally substituted C₃-C₈ cycloalkyl, optionally substituted C₂-C₈ heterocycloalkyl, optionally substituted aryl, or optionally substituted 5-, or 6-membered heteroaryl;
each R⁹ is independently hydrogen, halogen, —OR^{a}, —N(R^{a})₂, —SO₂N(R^{a})₂, —CO₂N(R^{a})₂, or optionally substituted alkyl; or 2 R⁹ on the same carbon are taken together to form an oxo; or 2 R⁹ on the adjacent carbons are taken together to form an alkene;
each R¹⁵ is independently hydrogen, halogen, —OR^{a}, —N(R^{a})₂, —SO₂N(R^{a})₂, —CO₂N(R^{a})₂, or optionally substituted alkyl;
each R^{a} is hydrogen or optionally substituted alkyl;
p is 0-4;
m is 0-3; and
X is a bond or C₁-C₆alkylene.

In some cases the auristatin amine has the following structure:

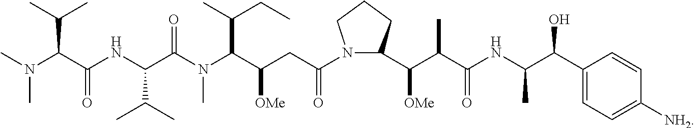

In some cases the antibody or fragment thereof binds an antigen on a cancer cell. In some cases the antibody or fragment thereof is an anti-CXCR4 antibody or fragment thereof. In some cases the phenyl group of the auristatin is linked to the antibody or fragment thereof via a linker. In some cases the linker comprises an amino acid. In some cases the amino acid is selected from a valine and a citrulline. In some cases the linker comprises a polyethylene glycol subunit. In some cases the auristatin and the linker together have a structure:

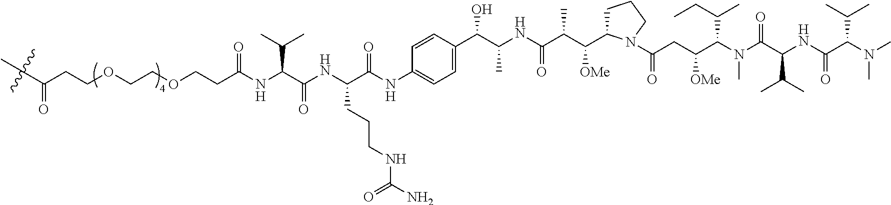

Further provided herein is a method of treating cancer or an autoimmune disease in a subject in need thereof, the method comprising administering an effective amount of the antibody drug conjugate to the subject.

In another aspect, provided herein is an antibody drug conjugate comprising a monomethyl auristatin linked to an antibody or fragment thereof through a non-cleavable linker.

In some cases the non-cleavable linker links the antibody or fragment thereof to a terminal amino group of monomethyl auristatin E.

In some cases the monomethyl auristatin E and the non-cleavable linker together have a structure:

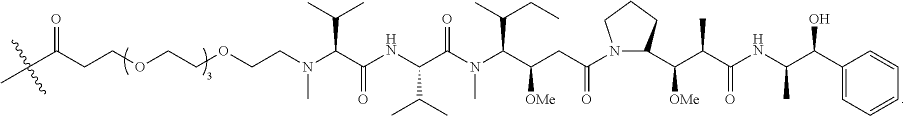

Also provided here is a method of treating cancer or an autoimmune disease in a subject in need thereof, the method comprising administering an effective amount of the antibody drug conjugate to the subject.

In another aspect, provided herein are methods of producing an antibody drug conjugate, the method comprising coupling an auristatin with a linker comprising an N-hydroxy-succinimide (NHS) ester. In some cases coupling the auristatin with the linker comprising the N-hydroxy-succinimide (NHS) ester produces a structure selected from:

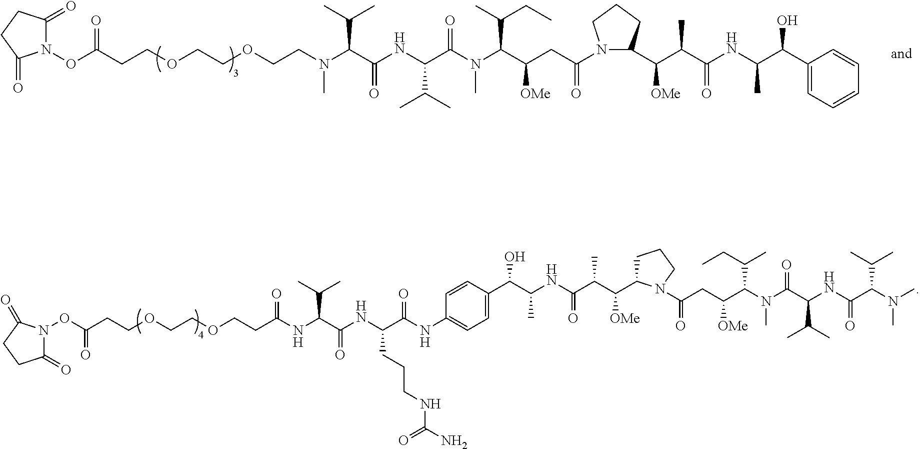

### BRIEF DESCRIPTION OF THE DRAWINGS

FIG. 1 shows construction of anti-CXCR4-auristatin conjugates. A schematic of non-specific conjugations of two auristatin drugs (indicated by asterisks) to anti-CXCR4 antibody (HLCX) in PBS (pH 7.4) with 10% DMSO via a reaction of NHS ester to surface-exposed lysine residues of the anti-CXCR4 antibody.

FIG. 2 shows various auristatins disclosed herein.

FIG. 3 shows characterization of anti-CXCR4 IgG antibody (HLCX) by SDS-PAGE analysis showing the intact antibody (left, no DTT) as well as the HC and LC (right) upon treatment with DTT.

FIG. 4 shows SDS-PAGE analysis of anti-CXCR4-auristatin conjugates with or without treatment with DTT; Lane 1=MW Marker, Lanes 2 and 4=anti-CXCR4 IgG-NC-MMAE, Lanes 3 and 5=anti-CXCR4 IgG-ValCit-MMAE.

FIGS. 5(A-D) show ESI-MS data of anti-CXCR4-auristatin conjugates after treatment with PNGase F (Promega) and 10 mM DTT with an estimated drug to antibody ratio (DAR) of ˜2-3. FIG. 5A shows MMAE conjugated by non-cleavable linker to the anti-CXCR4 IgG (HLCX) light chain. FIG. 5B shows MMAE conjugated by Val-Cit linker to the anti-CXCR4 IgG (HLCX) light chain. FIG. 5C shows MMAE conjugated by non-cleavable linker to the anti-CXCR4 IgG (HLCX) heavy chain. FIG. 5D shows MMAE conjugated by Val-Cit linker to the anti-CXCR4 IgG (HLCX) heavy chain.

FIG. 6 shows FACS analysis of binding of anti-CXCR4 IgG (HLCX) towards different cell lines. Ramos (CXCR4+), Jurkat (CXCR4+), and MDA-MB-231 (CXCR4−) cells were treated with 50 nM of either anti-CXCR4-AF488, 12G5-AF488 (positive control, 12G5=anti-CXCR4 antibody), or Her2-AF488 (negative control) for 1 h at 4° C. AF488=Alexa Fluor 488.

FIG. 7 shows cytotoxicity of Ramos (CXCR4+) cells after treatment with anti-CXCR4-auristatin conjugates, auristatin drugs, or antibody alone, with concentrations ranging from 600 nM to 30 pM for 4 days at 37° C. Cell viabilities were measured by addition of CellTiter-Glo (Promega) and the luminescent signals were read by EnVision plate reader.

FIG. 8 shows cytotoxicity of Jurkat (CXCR4+) cells after treatment with anti-CXCR4-auristatin conjugates, small molecule drugs, or antibody alone, with concentrations ranging from 600 nM to 30 pM for 4 days at 37° C. Cell viabilities were measured by addition of CellTiter-Glo (Promega) and the luminescent signals were read by EnVision plate reader.

FIG. 9. shows cytotoxicity of MDA-MB-231 (CXCR4−) cells after treatment with anti-CXCR4-auristatin conjugates, small molecule drugs, or antibody alone, with concentrations ranging from 600 nM to 30 pM for 4 days at 37° C. Cell viabilities were measured by addition of CellTiter-Glo (Promega) and the luminescent signals were read by EnVision plate reader.

FIG. 10 shows cytotoxicity of various auristatins in Ramos cells.

FIG. 11 shows cytotoxicity of various auristatins in Jurkat cells.

FIG. 12 shows cytotoxicity of various auristatins in SKBR3 cells.

FIG. 13 shows cytotoxicity of various auristatins in MDA-MB-231 cells.

FIG. 14 shows cytotoxicity of various auristatins in OPM-2 cells.

FIG. 15 shows cytotoxicity of various auristatins in CHO—S cells.

FIG. 16 shows characterization of the anti-CXCR4 IgG antibody HLCX by ESI-MS analysis that corroborates the result of the SDS-PAGE analysis showing the molecular weights of both the HC and LC of the antibody after treatment with PNGase F (Promega, 12 h at 37° C. in PBS pH 7.4) and subsequent reduction of the disulfide bonds with 10 mM DTT: LC Calc. MW=23451 Da, Observed MW=23454 Da, also observed part of signal sequence; HC Calc. MW=51758 Da, Observed MW=51761 Da. DTT=dithiothreitol, HC=heavy chain, LC=light chain, ESI-MS=electrospray ionization mass spectrometry. Figure discloses “LVTNS” as SEQ ID NO: 5.

FIG. 17 shows a synthesis scheme for NHS—NC-MMAE.

FIG. 18 shows a synthesis scheme for auristatin E amine.

FIG. 19 shows the structures of NHS—NC-MMAE or NHS-ValCit-MMAE.

### DETAILED DESCRIPTION OF THE INVENTION

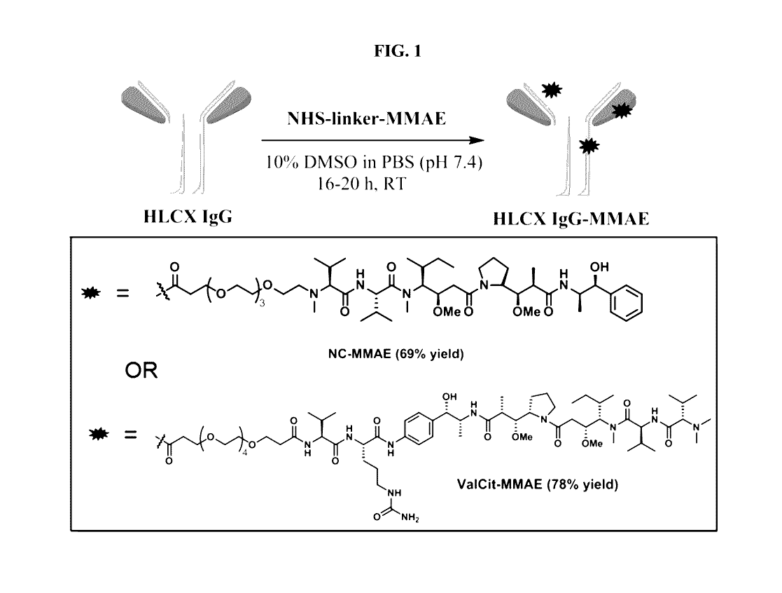

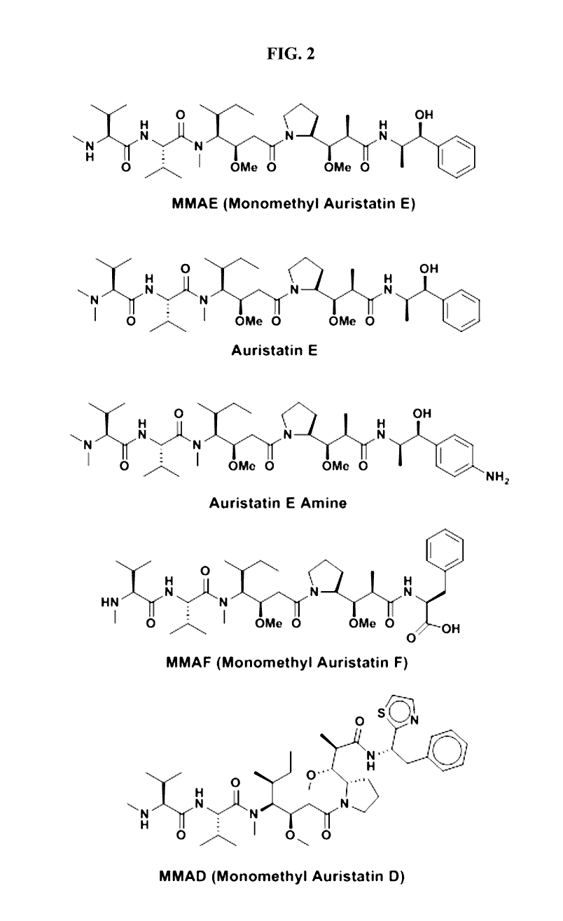

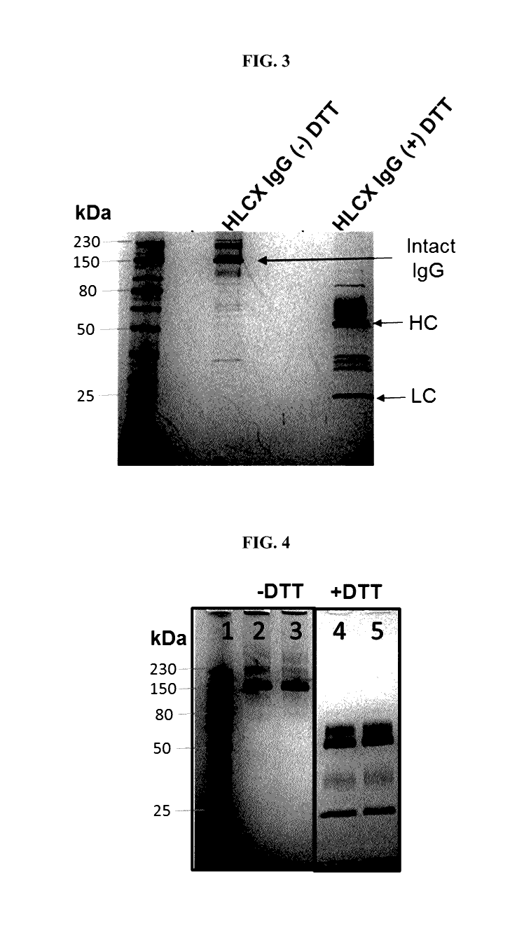

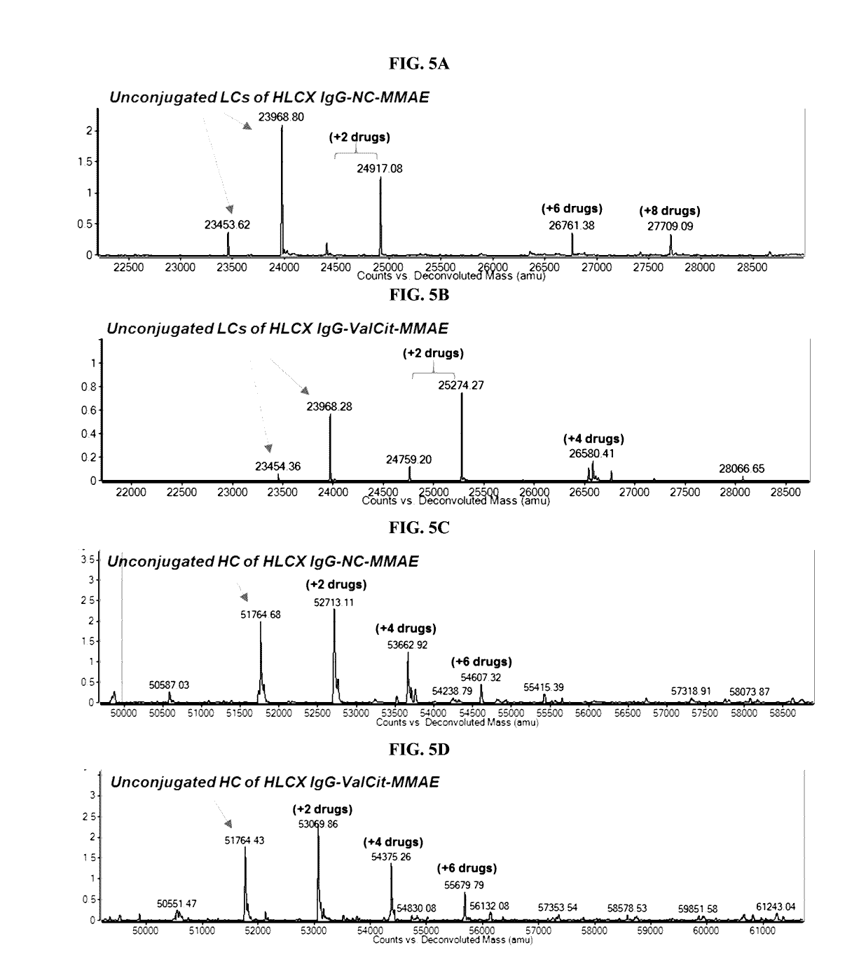

Disclosed herein are auristatin-antibody conjugates. In many cases, toxicity of auristatin is overcome or reduced via the antibody linkage, due to the specificity of the antibody for an antigen on a target cell. Because of the antimitotic and antineoplastic activities of auristatins, the antibody portion of the auristatin-antibody conjugate is generally specific for an antigen of a cancer cell. In many cases, auristatin-antibody conjugates disclosed herein are highly potent and differ from previously known auristatin-antibody conjugates by the use of novel linkage sites on the auristatin molecule, enabling the use of both novel cleavable and non-cleavable linkers (see FIG. 1 for examples of auristatin and linker combinations). A novel derivative of auristatin disclosed herein, specifically auristatin amine, allows for use of these linkers and novel methods of producing conjugates.

Disclosed herein are antibody drug conjugates comprising: an auristatin; and an antibody or fragment thereof, wherein the antibody or fragment thereof is linked to a phenyl group of the auristatin. The auristatin may be selected from auristatin D, auristatin E, auristatin F, monomethyl auristatin D, monomethyl auristatin E, and monomethyl auristatin F. The auristatin may be an auristatin amine. The auristatin may have the structure:

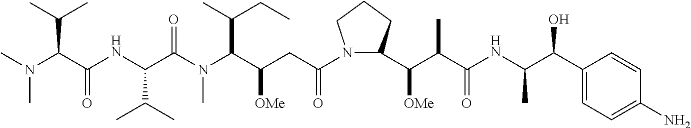

The antibody or fragment thereof may bind an antigen on a cancer cell. The antibody or fragment thereof may be an anti-CXCR4 antibody or fragment thereof. The antibody or fragment thereof may be an anti-CD74 antibody or fragment thereof. The antibody drug conjugate may further comprise a linker that links the auristatin to the antibody or fragment thereof. The linker may comprise an amino acid. The amino acid may be selected from a valine and a citrulline. The linker may comprise a polyethylene glycol subunit. The auristatin and the linker together may have the structure:

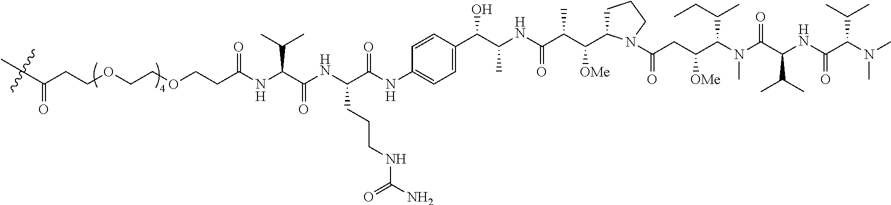

Further disclosed herein are antibody drug conjugate comprising: auristatin; an anti-CXCR4 antibody or fragment thereof; and a non-cleavable linker that links the auristatin to the antibody or fragment thereof.

Disclosed herein are antibody drug conjugates comprising: monomethyl auristatin E; an antibody or fragment thereof; and a non-cleavable linker that links the monomethyl auristatin E to the antibody or fragment thereof. The antibody or fragment thereof may be an anti-CXCR4 antibody. The antibody or fragment thereof may be an anti-CD74 antibody. The non-cleavable linker may link the antibody or fragment thereof to a terminal amino group of the auristatin. The auristatin and the linker together may have the structure:

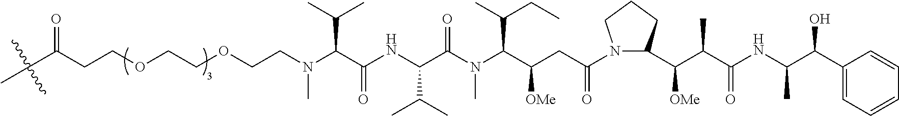

Further disclosed herein are antibody drug conjugates comprising: monomethyl auristatin E; and an anti-CXCR4 antibody or fragment thereof, wherein the antibody or fragment thereof is linked to a phenyl group of the auristatin.

Disclosed herein are antibody drug conjugates comprising: monomethyl auristatin E; and an anti-CD74 antibody or fragment thereof, wherein the antibody or fragment thereof is linked to a phenyl group of the auristatin.

Further disclosed herein are methods of producing the auristatin-antibody conjugate disclosed herein, wherein the method comprises coupling the auristatin with a linker comprising a N-hydroxy-succinimide (NHS) ester at a first end of the linker. The coupling the auristatin with the linker may comprise the N-hydroxy-succinimide (NHS) ester at one end produces a structure selected from:

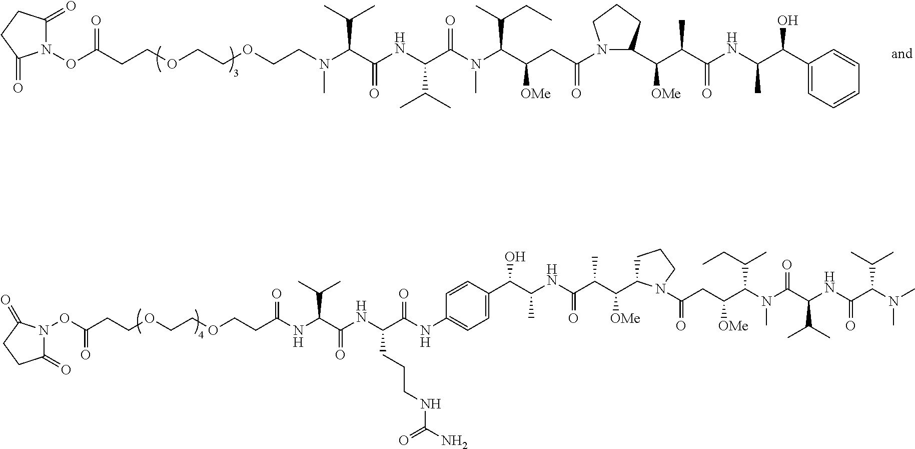

The method may further comprise coupling a second end of the linker to the antibody or fragment thereof.

Disclosed herein are methods of treating a disease or condition in a subject in need thereof comprising administering any one of the antibody drug conjugates disclosed herein to the subject. The disease or condition may be a cancer. The disease or condition may be an autoimmune disease.

Further disclosed herein are compounds having the structure:

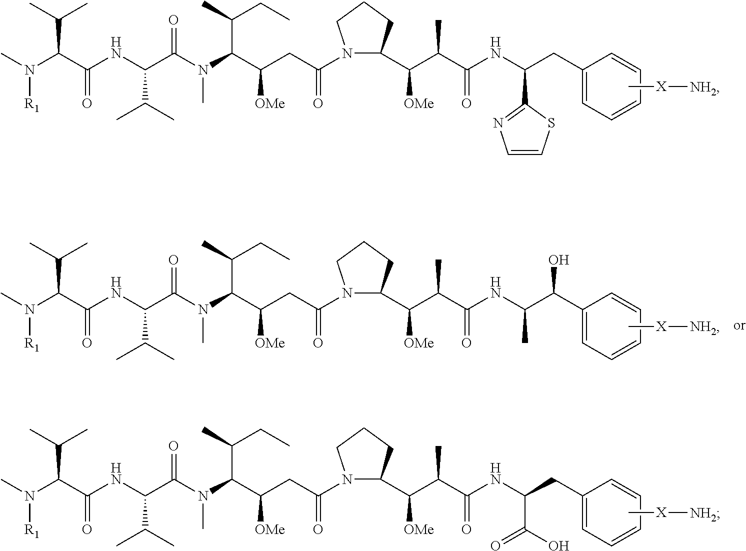

wherein R₁ is H or CH₃; and X is a bond or C₁-C₆alkyl.

The compound may have the structure:

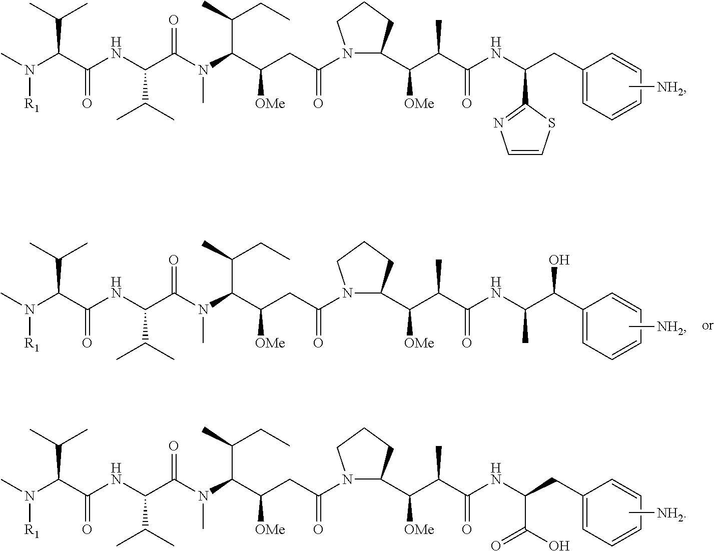

The compound may have the structure:

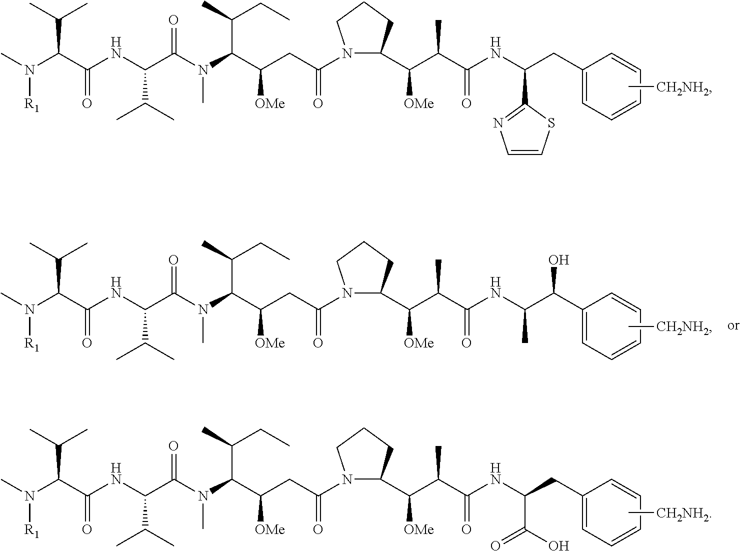

The compound may have the structure:

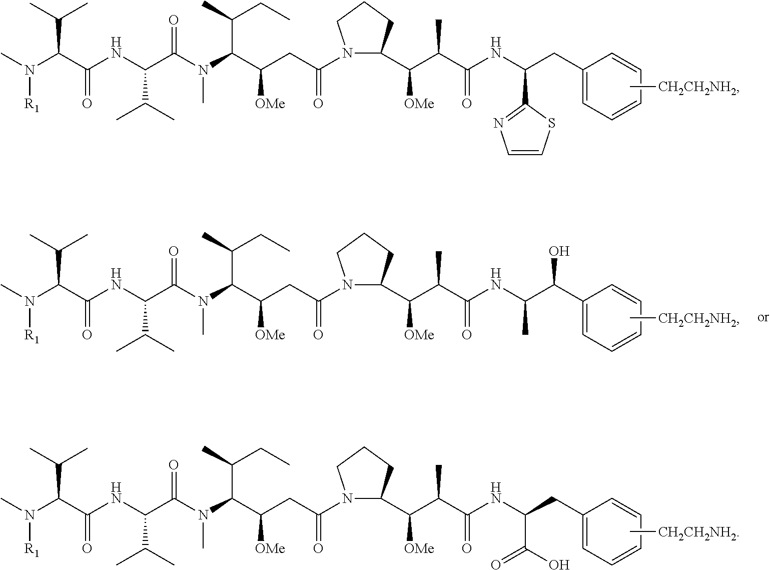

The compound may have the structure:

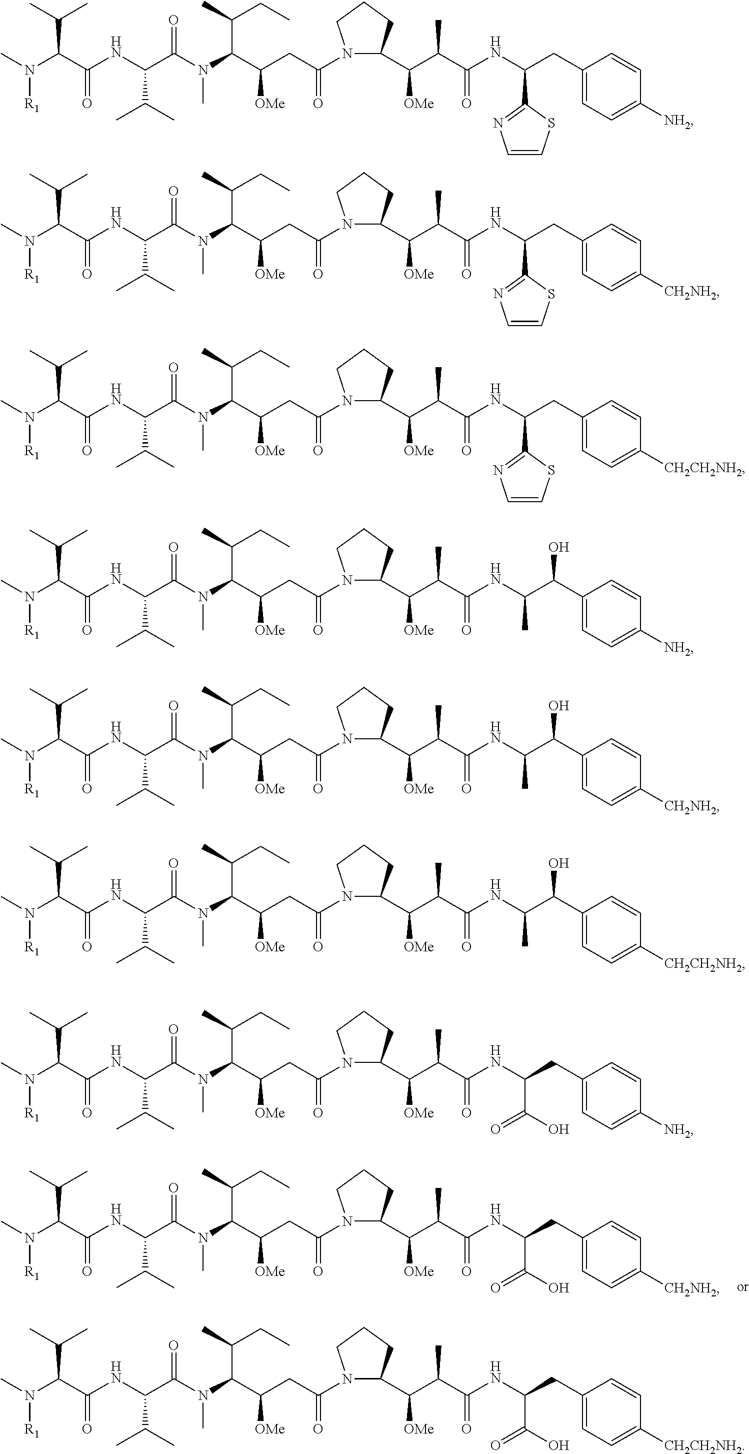

Disclosed herein are compounds having the structure:

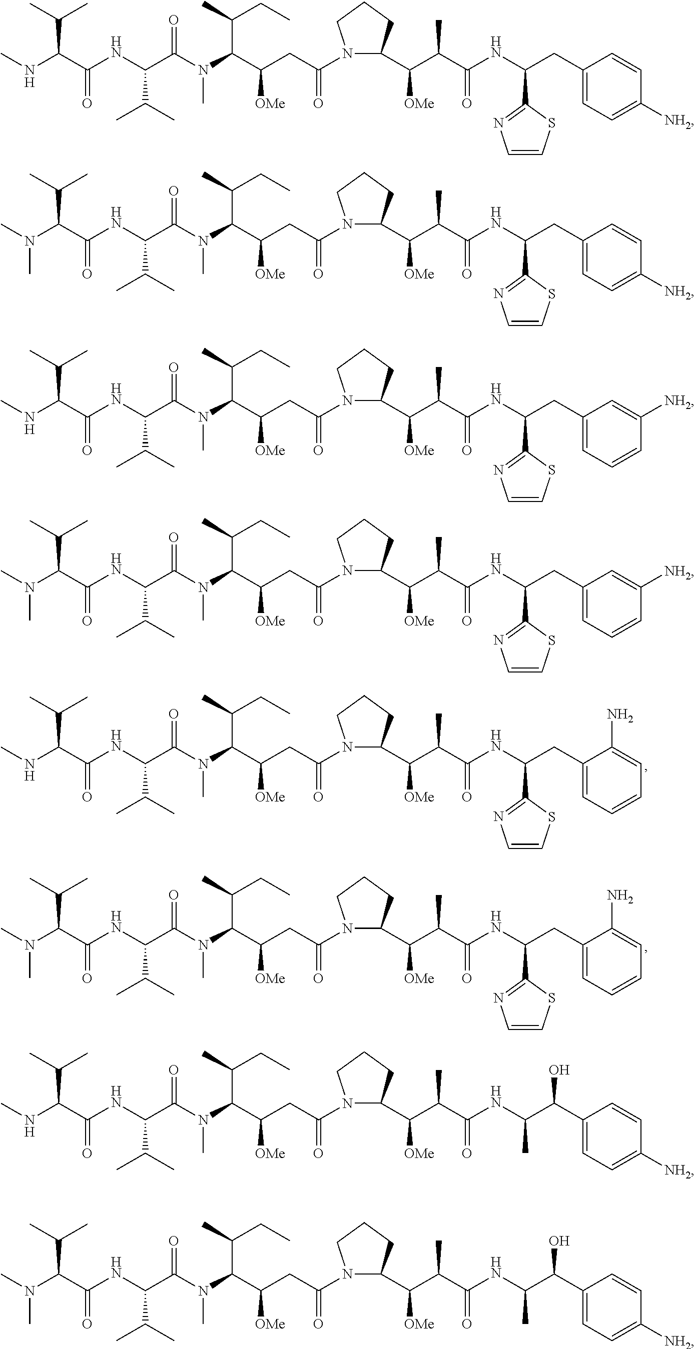

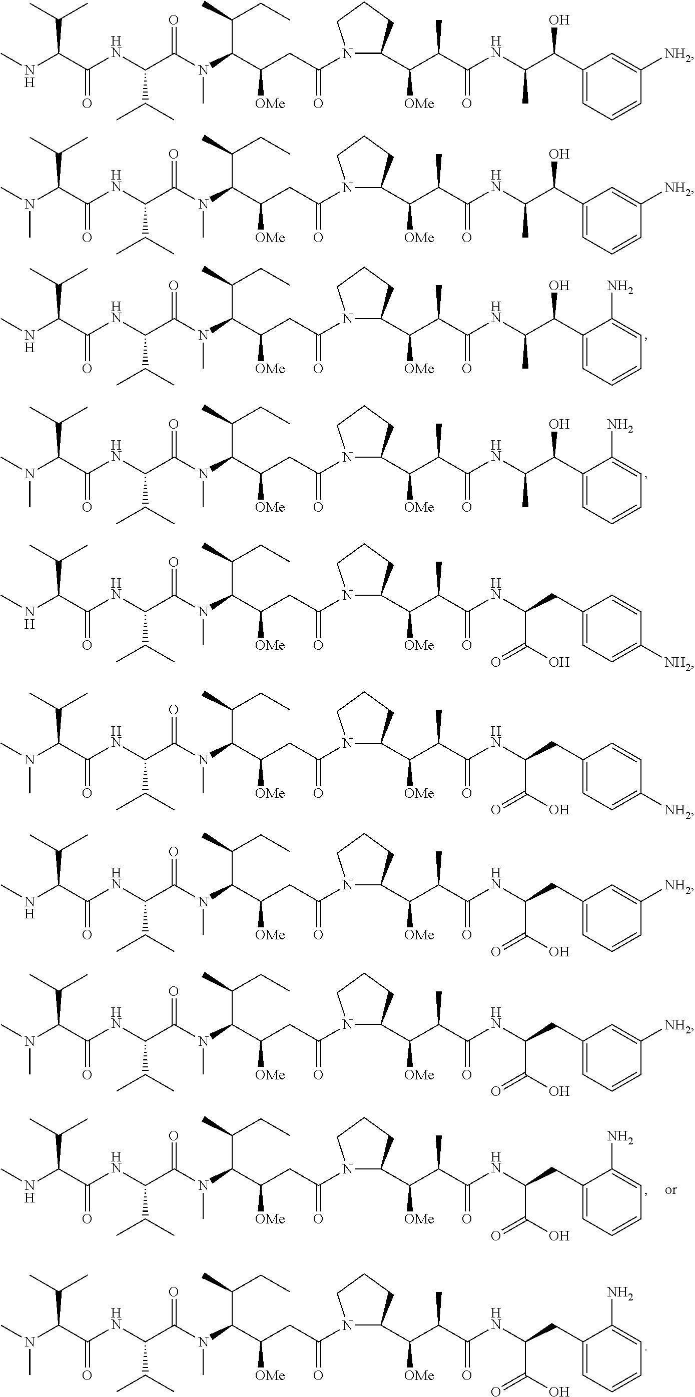

### Auristatin-Antibody Conjugates

Disclosed herein are auristatin-antibody conjugates that comprise an auristatin and an antibody or antibody fragment, wherein the antibody or antibody fragment is linked to the phenyl group of the auristatin. Generally, the auristatin-antibody conjugates comprise a linker that links the auristatin and the antibody or antibody fragment. Further disclosed herein are auristatin-antibody conjugates that comprise an auristatin and an antibody or antibody fragment, wherein the antibody or antibody fragment is linked by a non-cleavable linker.

The auristatin-antibody conjugates may comprise a single auristatin. The auristatin-antibody conjugates may comprise a plurality of auristatins. The auristatin-antibody conjugates may comprise about 1, about 2, about 3, about 4, about 5, about 6, about 7, about 8, about 9, about 10 or more auristatins. The auristatin-antibody conjugates may comprise a plurality of auristatins comprising a first auristatin and a second auristatin. The first auristatin and the second auristatin may be the same. The first auristatin and the second auristatin may be different.

The auristatin-antibody conjugates may have a half maximal inhibitory concentration for killing cells (IC₅₀). The IC₅₀ may be less than about 100 nM, less than about 80 nM, less than about 60 nM, less than about 40 nM, less than about 20 nM, or less than about 10 nM. The IC₅₀ may be less than about 9.5 nM, less than about 9 nM, less than about 8.5 nM, less than about 8 nM, less than about 7.5 nM, less than about 7 nM, less than about 6.5 nM, less than about 6 nM, less than about 5.5 nM, less than about 5 nM, less than about 4.5 nM, less than about 4 nM, less than about 3.5 nM, less than about 3 nM, less than about 2.5 nM, less than about 2 nM, less than about 1.5 nM, less than about 1 nM, or less than about 0.5 nM. The IC₅₀ may be less than about 10 nM. The IC₅₀ may be less than about 5 nM. The IC₅₀ may be less than about 2 nM. The IC₅₀ may be less than about 1 nM.

### Auristatin

Disclosed herein are auristatin-antibody conjugates that comprise an auristatin or an auristatin amine. Auristatin may be a compound of Formula (I):

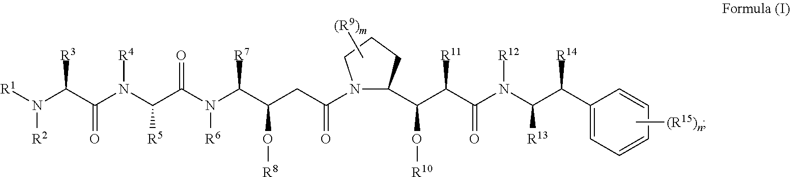

wherein
each R¹, R², R⁴, R⁶, and R¹² is independently hydrogen or optionally substituted alkyl;
each R³, R⁵, R⁷, and R¹¹ is independently optionally substituted alkyl;
each R⁸ and R¹⁰ is independently optionally substituted alkyl;
each R¹³ and R¹⁴ are independently hydrogen, halogen, —CN, —OR^{a}, —COOR^{a}, —N(R^{a})₂, optionally substituted alkyl, optionally substituted C₃-C₈ cycloalkyl, optionally substituted C₂-C₈ heterocycloalkyl, optionally substituted aryl, or optionally substituted 5-, or 6-membered heteroaryl;
each R⁹ is independently hydrogen, halogen, —OR^{a}, —N(R^{a})₂, —SO₂N(R^{a})₂, —CO₂N(R^{a})₂, or optionally substituted alkyl; or 2 R⁹ on the same carbon are taken together to form an oxo; or 2 R⁹ on the adjacent carbons are taken together to form an alkene;
each R¹⁵ is independently hydrogen, halogen, —OR^{a}, —N(R^{a})₂, —SO₂N(R^{a})₂, —CO₂N(R^{a})₂, or optionally substituted alkyl;
each R^{a} is hydrogen or optionally substituted alkyl;
n is 0-5; and
m is 0-3.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁵ is methyl; R¹¹ is methyl; R¹² is hydrogen; m is 0; n is 1-5; each R¹⁵ is independently hydrogen, halogen, —OR^{a}, —N(R^{a})₂, —SO₂N(R^{a})₂, —CO₂N(R^{a})₂, or optionally substituted alkyl; and R^{a} is hydrogen or optionally substituted alkyl.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁻° is methyl; R¹¹ is methyl; R¹² is hydrogen; m is 0; n is 0; R¹ is methyl; R¹³ is methyl; and R¹⁴ is —OH.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; is hydrogen; R¹³ is methyl; and R¹⁴ is —OH.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is methyl; R¹³ is —COOH; and R¹⁴ is hydrogen.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; is hydrogen; R¹³ is —COOH; and R¹⁴ is hydrogen.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 0; R¹ is methyl; R¹³ is 2-thiazolyl; and R¹⁴ is hydrogen.

Auristatin may be a compound of Formula (I) wherein R² is methyl; R³ is isopropyl; R⁴ is hydrogen; R⁵ is isopropyl; R⁶ is methyl; R⁷ is 2-butyl; R⁸ is methyl; R⁹ is hydrogen; R¹⁰ is methyl; R¹¹ is methyl; R¹² is hydrogen; m is 0; n is 1; is hydrogen; R¹³ is 2-thiazolyl; and R¹⁴ is hydrogen.

Also disclosed herein are auristatin amines. The auristatin amine may be a compound of Formula (II):

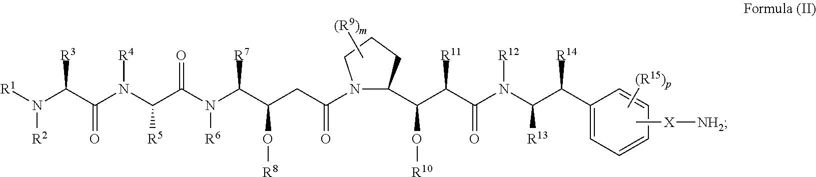

wherein
each R¹, R², R⁴, R⁶, and R¹² is independently hydrogen or optionally substituted alkyl;
each R³, R⁵, R⁷, and R¹¹ is independently optionally substituted alkyl;
each R⁸ and R¹⁰ is independently optionally substituted alkyl;
each R¹³ and R¹⁴ are independently hydrogen, halogen, —CN, —OR^{a}, —COOR^{a}, —N(R^{a})₂, optionally substituted alkyl, optionally substituted C₃-C₈ cycloalkyl, optionally substituted C₂-C₈ heterocycloalkyl, optionally substituted aryl, or optionally substituted 5-, or 6-membered heteroaryl;
each R⁹ is independently hydrogen, halogen, —OR^{a}, —N(R^{a})₂, —SO₂N(R^{a})₂, —CO₂N(R^{a})₂, or optionally substituted alkyl; or 2 R⁹ on the same carbon are taken together to form an oxo; or 2 R⁹ on the adjacent carbons are taken together to form an alkene;
each R¹⁵ is independently hydrogen, halogen, —OR^{a}, —N(R^{a})₂, —SO₂N(R^{a})₂, —CO₂N(R^{a})₂, or optionally substituted alkyl;
each R^{a} is hydrogen or optionally substituted alkyl;
p is 0-4;
m is 0-3; and
X is a bond or C₁-C₆alkylene.

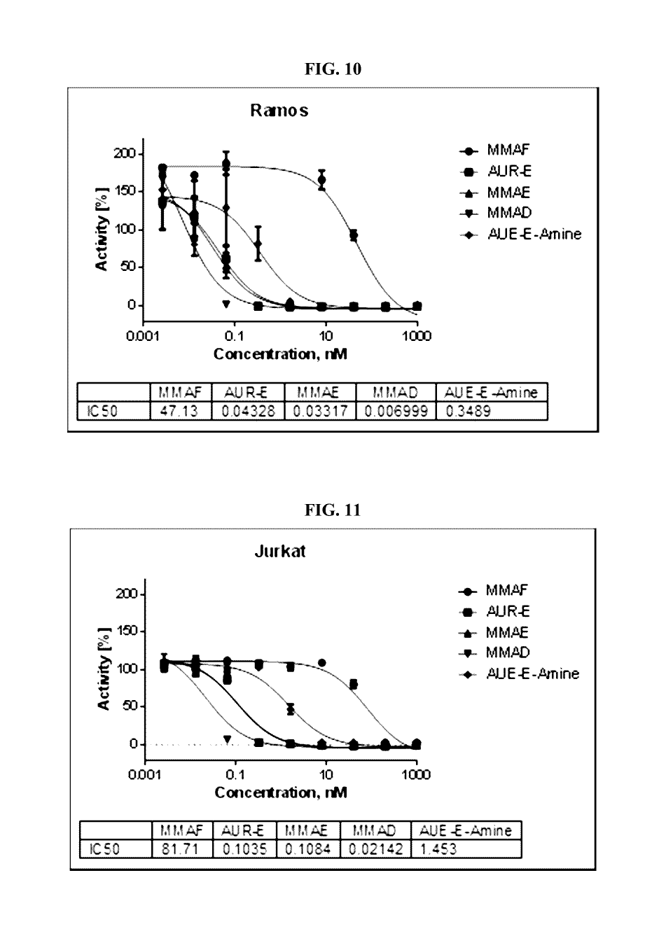

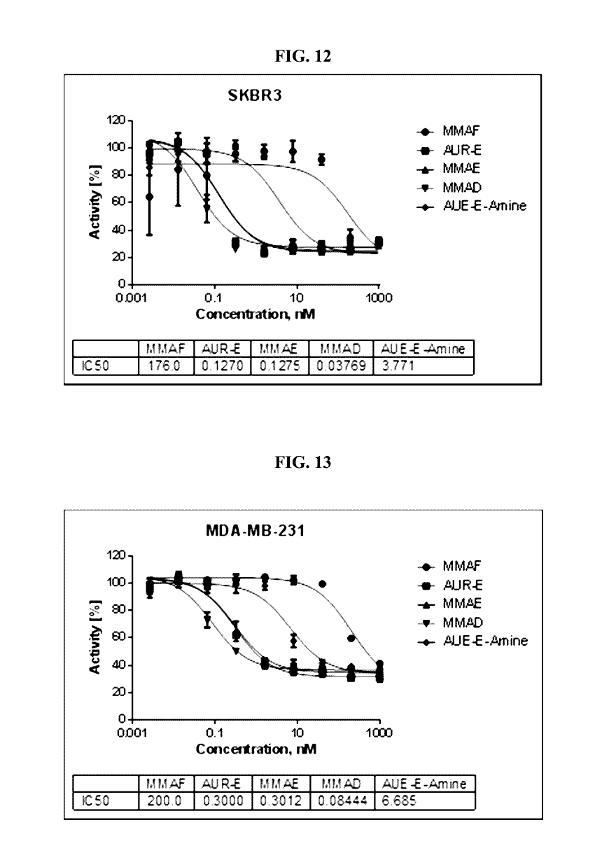

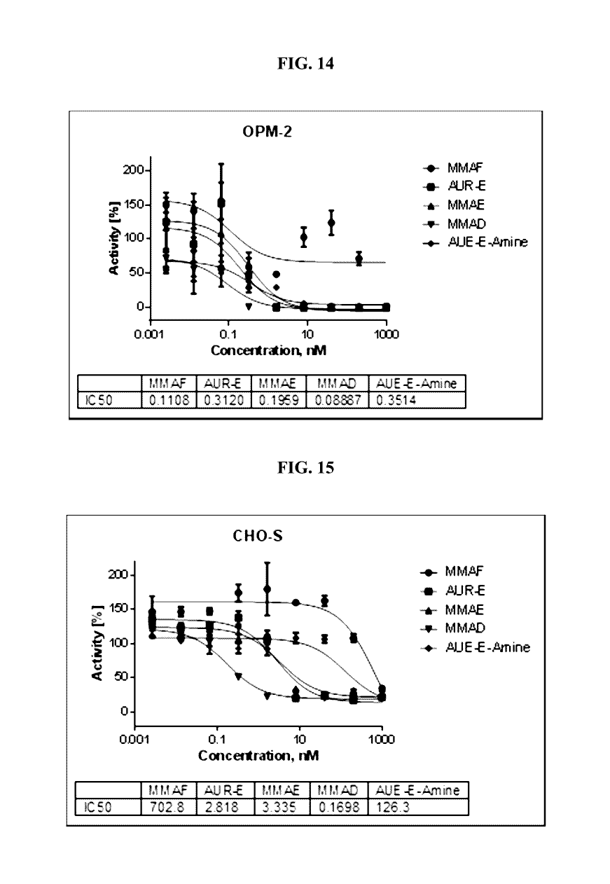

The amine on the auristatin amine may be utilized as a conjugation site to the antibody or antibody fragment. The amine on the auristatin amine may be linked to the antibody or antibody fragment thereof via a linker. The auristatin amine may be a monomethyl auristatin amine. The auristatin amine may have equipotent or similar potency as previously known auristatins, monomethyl auristatins and other derivatives thereof (see, e.g., cytotoxic activities of auristatins for various cells as shown in FIGS. 10-15).

The auristatin amine described herein may have the following structure:

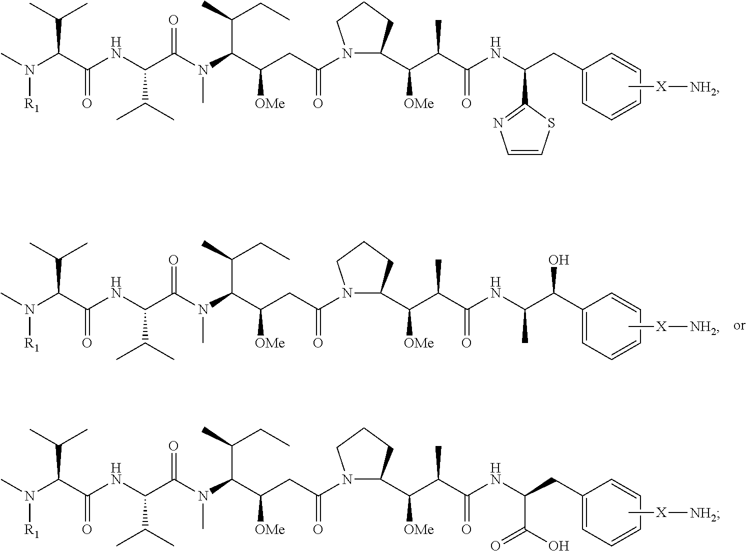

wherein R₁ is H or CH₃; and X is a bond or C₁-C₆alkylene.

The auristatin amine described herein may have the following structure:

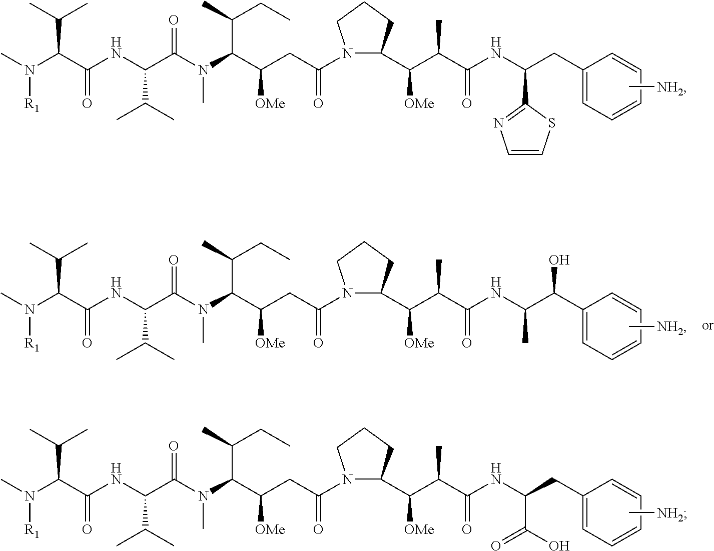

wherein R₁ is H or CH₃.

The auristatin amine described herein may have the following structure:

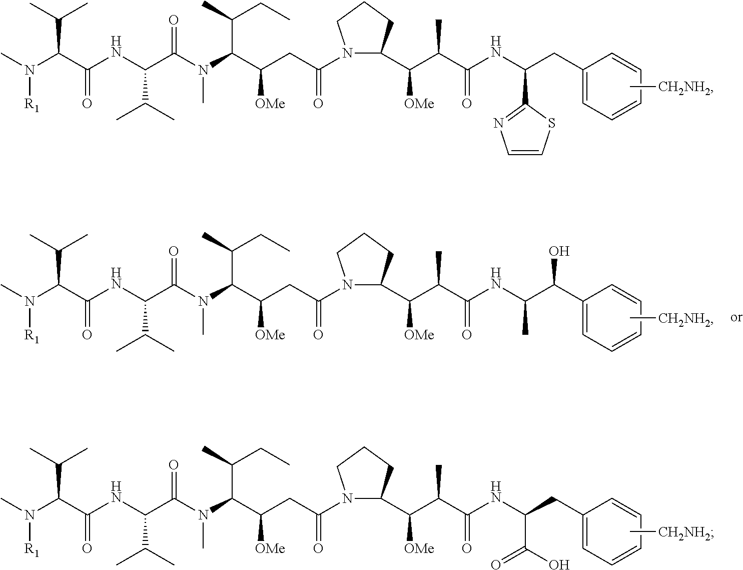

wherein R₁ is H or CH₃.

The auristatin amine described herein may have the following structure:

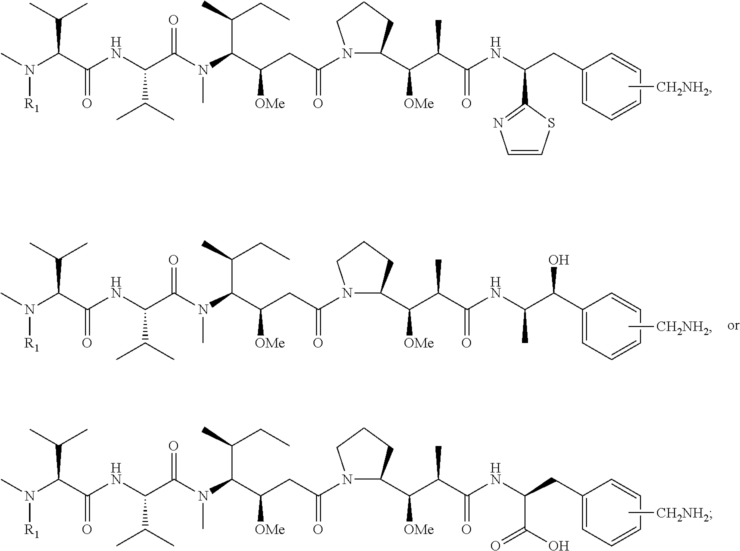

wherein R₁ is H or CH₃.

The auristatin amine described herein may have the following structure:

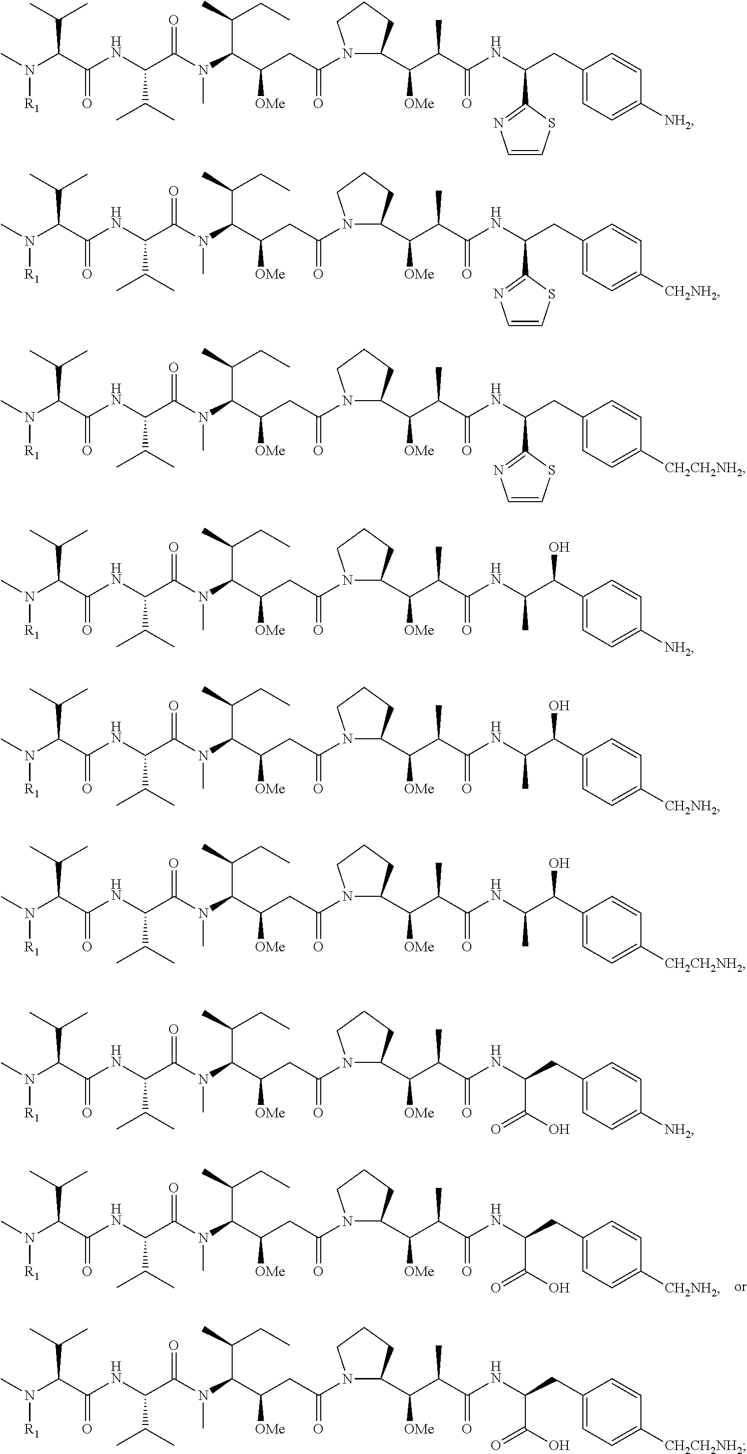

wherein R₁ is H or CH₃.

The auristatin amine described herein may have the following structure:

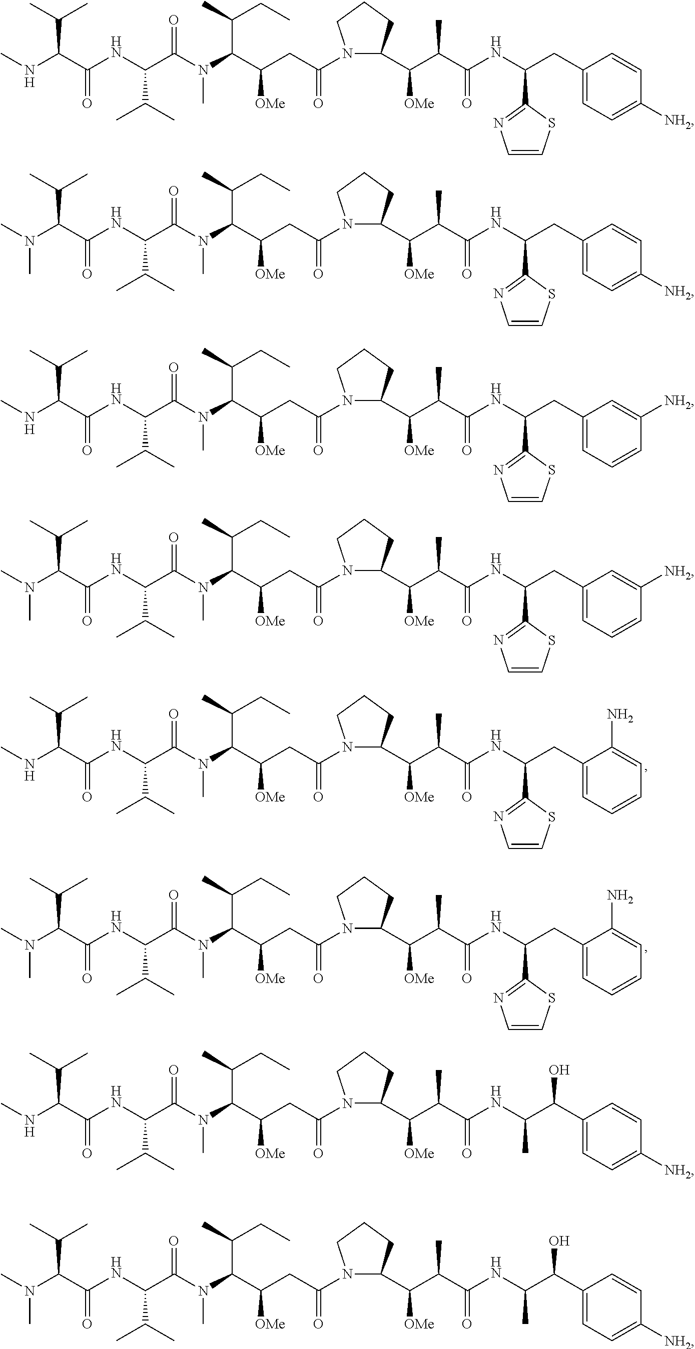

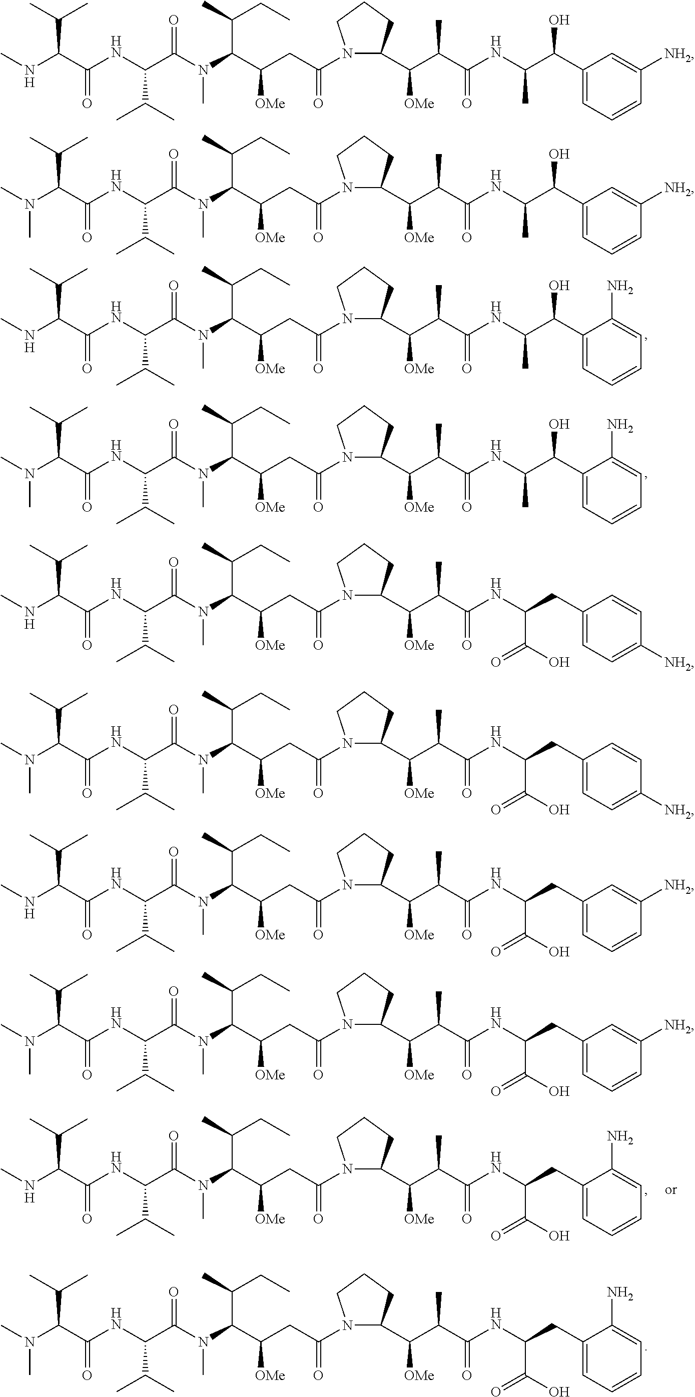

Auristatin may be selected from, but are not limited to, auristatin D, auristatin E, and auristatin F. The auristatin may be a desmethyl-auristatin or monomethyl auristatin. The monomethyl auristatin may be monomethyl auristatin D. The monomethyl auristatin may be monomethyl auristatin E. The monomethyl auristatin may be monomethyl auristatin F. In various cases, the auristatin, auristatin amine, or monomethyl auristatin retains an antimitotic and/or antineoplastic activity. In many cases, an auristatin blocks polymerization of tubulin.

### Antibodies

Disclosed herein are auristatin-antibody conjugates that comprise an antibody or antibody fragment that binds an antigen. The antibody or antibody fragment may be a chimeric, recombinant, engineered, synthetic, humanized, fully human, or human engineered antibody. The antibody or antibody fragment may be monoclonal or polyclonal. The antibody may be a bispecific antibody. The antibody or antibody fragment may be chemically modified in order to increase its serum half-life.

The antibody may comprise at least a portion of an antibody or an entire antibody. The antibody may be an IgG. The antibody or antibody fragment may comprise at least a portion of a heavy chain, a portion of a light chain, a portion of a variable region, a portion of a constant region, a portion of a complementarity determining region (CDR), or a combination thereof. The antibody or antibody fragment may comprise at least a portion of the Fc (fragment, crystallizable) region. The antibody or antibody fragment may comprise may comprise at least a portion of the complementarity determining region (e.g., CDR1, CDR2, CDR3). The antibody or antibody fragment may comprise at least a portion of a Fab (fragment, antigen-binding) region. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The antibody may be selected from gemtuzumab, alemtuzumab, trastuzumab, rituximab; cetuximab erbitux; nimotuzumab, bevacizumab, and bavituximab. The antibody may be an anti-CXCR4 antibody or fragment thereof. The antibody may be an anti-CD74 antibody.

The anti-CXCR4 antibody may be HLCX. HLCX may have a heavy chain with a sequence of SEQ ID NO. 1. The antibody may be HLCX. HLCX may have a heavy chain with a sequence that is at least about 90% homologous or identical to that of SEQ ID NO. 1. HLCX may have a light chain with a sequence of SEQ ID NO. 2. HLCX may have a light chain with a sequence that is at least about 90% homologous or identical to that of SEQ ID NO. 2.

The antigen may be a tumor antigen. Tumor antigens (or neoantigens) may be antigens that are presented by MHC I or MHC II molecules on the surface of tumor cells. These antigens may sometimes be presented by tumor cells and never by corresponding normal/benign cells. In this case, they are called tumor-specific antigens (TSAs) and, in general, result from a tumor-specific mutation. More common are antigens that are presented by tumor cells and normal cells, and they are called tumor-associated antigens (TAAs). Tumor antigens may be on the surface of the tumor in the form of, for example, a mutated receptor.

The antigen may be more highly expressed on a malignant cell relative to a benign cell. The antigen may have an expression level on a malignant cell that is at least about 20%, at least about 30%, at least about 40%, at least about 50%, at least about 60%, at least about 70%, at least about 80%, at least about 90%, or at least about 100% greater than an expression level on a benign cell. The antigen may have an expression level on a malignant cell that is at least about 2-fold, at least about 3-fold, at least about 4-fold, at least about 5-fold, at least about 6-fold, at least about 7-fold, at least about 8-fold, at least about 9-fold, or at least about 10-fold greater than an expression level on a benign cell. The antigen may have an expression level on a malignant cell that is at least about 20-fold, at least about 30-fold, at least about 40-fold, at least about 50-fold, at least about 60-fold, at least about 70-fold, at least about 80-fold, at least about 90-fold, or at least about 100-fold greater than an expression level on a benign cell.

The antigen may be selected from GD2, B7-H3, CA-125, CD51, EpCAM, VEGFR, mesothelin, activin receptor-like kinase 1, phosphatidylserine, BAFF, CD44, CD274, CD19, CD22, CD20, EGFR, Her2, BCMA, CS1, CLL, CEA, CD30, MUC1, Lewis-Y antigen, IGF-1 receptor, glypican 3, TRAIL-R1, TRAIL-R2, CD40, insulin-like growth factor receptor, CD38, DLL4, RANKL, histone complex, DRS, ERBB3, SLAMF7, CSF1R, HHGFR, TWEAK receptor, B7-H3, SAC, CD3, an integrin, folate receptor, TYRP1, CD80, CD33, carbonic anhydrase 9, GPNMB, CLDN18.2, SDC1, GUCY2C, CD51, CD152, CD38, PDCD1, phosphate-sodium co-transporter, CD37, CD56, CD40, CD23, ch4D5, GD3 ganglioside, CCR4, C242 antigen, 5T4, RON, angiopoietin 2, PDGF receptor, TEM1, EGFL7, HER3, PDCD1, PD-1, adenocarcinoma antigen, CD79B, vimentin, fibronectin, tumor-associated calcium signal transducer 2, CD200, TAG-72, FAP, BAFF, alpha-fetoprotein, Notch receptor, tanascin C, CD221, CD28, CTLA-4, CD140a, MS4A1, 4-1BB, STEAP1, Frizzled receptor, CD70, tumor antigen CTAA16.88, CD4, MAGE-1, MAGE-2, MAGE-3, MAGE-4, anti-transferrin receptor, p97, MUC1-KLH, gp100, MART1, PSA, PSMA, IL-2 receptor, CD52, mucin, P21, MPG, Neu oncogene product, and Her1. The antigen may be CD74.

The antigen may be CXCR4. C—X—C chemokine receptor type 4 (CXCR-4) also known CD184 (cluster of differentiation 184) an alpha-chemokine receptor that binds stromal-derived-factor-1 (SDF-1 also called CXCL12), which has potent chemotactic activity for lymphocytes, as well as macrophage migration inhibitory factor (MIF). CXCR4 expression is usually low or absent in healthy tissues, its role being more predominant during embryotic development. However, expression has been found to be elevated in many cancers, including, but not limited to, breast cancer, ovarian cancer, melanoma, and prostate cancer, Waldenstrom's macroglobulinemia, and B-cell malignancy. In addition, high levels of CXCL12 expression in lung, liver and bone marrow correlates with metastasis to these tissues.

### Linkers

Disclosed herein are auristatin-antibody conjugates that comprise a linker that links auristatin to the antibody. The auristatin-antibody conjugate may comprise a plurality of linkers that link a plurality of auristatins to the antibody or antibody fragment. The linker may be cleavable. The linker may not be cleavable (also referred to herein as non-cleavable (NC)). The linker may link the antibody to an amino acid of the auristatin. The linker may link the antibody to a non-amino acid group of the auristatin. The linker may link the antibody to a phenyl group of the auristatin. The linker may link the antibody to a terminal phenyl group of the auristatin. The linker may link the antibody to the phenyl group of auristatin E. The linker may link the antibody to the phenyl group of monomethyl auristatin E. The linker may link the antibody to the phenyl group of auristatin F. The linker may link the antibody to the phenyl group of monomethyl auristatin F. The linker may link the antibody to the phenyl group of auristatin D. The linker may link the antibody to the phenyl group of monomethyl auristatin D. The linker may link the antibody to the para position carbon (C4) of the phenyl group of auristatin. The linker may link the antibody to the para position carbon (C2) of the phenyl group of auristatin. The linker may link the antibody to the para position carbon (C3) of the phenyl group of auristatin.

The linker may link the auristatin to a terminus of the antibody or antibody fragment. The linker may link the auristatin to a C terminus of the antibody or antibody fragment. The linker may link the auristatin to an N terminus of the antibody or antibody fragment. The linker may link the auristatin to a region of the antibody or antibody fragment selected from a variable region, a constant region, a light chain, a heavy chain, a CDR, and a hinge region. The CDR may be selected from CDR1, CDR2 and CDR3. The region of the antibody or antibody fragment may be a loop on the CDR. The region of the antibody or antibody fragment may be selected from a CH1 domain, a CH2 domain, a CH3 domain, a CL domain, a VH domain, and a VL domain. The linker may link the auristatin to a C terminus of a CL domain or a C terminus of a CH domain. The linker may link the auristatin to an N terminus of a VH domain or an N terminus of a VL domain. The linker may link the auristatin to a C terminus of the heavy chain. The linker may link the auristatin to a C terminus of the light chain. The linker may link the auristatin to an N terminus of the heavy chain. The linker may link the auristatin to an N terminus of the light chain.

The linker may link the auristatin to a lysine of the antibody or antibody fragment. The plurality of linkers may link the plurality of auristatins to a plurality of lysines on the antibody or antibody fragment. The plurality of lysines may be on the light chain of the antibody or antibody fragment. The plurality of lysines may be on the heavy chain of the antibody or antibody fragment. The plurality of lysines may be on the light chain and the heavy chain of the antibody or antibody fragment.

The auristatin antibody conjugate may comprise at least about 1 linker, at least about 2 linkers, at least about 3 linkers, at least about 4 linkers, at least about 5 linkers, at least about 6 linkers, at least about 7 linkers, at least about 8 linkers, at least about 9 linkers, or at least about 10 linkers. Thus 1 or more, 2 or more, 3 or more, 4 or more, 5 or more, 6 or more, 7 or more, 8 or more, 9 or more, or 10 or more auristatins may be linked to the antibody or antibody fragment.

The linker may comprise polyethylene glycol. The linker may comprise one or more polyethylene glycol subunits. The linker may comprise at least 1, at least 2, at least 3, or at least 4 polyethylene glycol subunits. The linker may comprise an amino acid. The linker may comprise a plurality of amino acids. The amino acid(s) may be selected from any natural or unnatural amino acid. The amino acid may be valine. The amino acid may be citrulline. The amino acids may be valine and citrulline. The amino acids valine and citrulline in the linker may provide a cleavage site. The cleavage site may be recognized and cleaved by cathepsin B. Other amino acids and combinations thereof, such as phenylalanine and lysine, by way of non-limiting example, may be used as well.

The linker may link the antibody or antibody fragment to the amine group of the auristatin amine. The linker may link the antibody or antibody fragment to the amine group of the auristatin E amine.

### Pharmaceutical Compositions

Disclosed herein are pharmaceutical compositions that comprise auristatin-antibody conjugates disclosed herein. The pharmaceutical composition may comprise one or more pharmaceutically acceptable salts, excipients or vehicles. Pharmaceutically acceptable salts, excipients, or vehicles include, but are not limited to, carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants. Neutral buffered saline or saline mixed with serum albumin are exemplary appropriate carriers.

The compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form, lyophilized form or freeze-dried form. The composition may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, antimicrobials, antioxidants, chelating agents, inert gases and the like. See generally, Remington's Pharmaceutical Science, 16th Ed., Mack Eds., 1980, which is incorporated by reference in its entirety.

Pharmaceutical compositions described herein may be formulated for controlled or sustained delivery in a manner that provides local concentration of the product (e.g., bolus, depot effect) and/or increased stability or half-life in a particular local environment. The compositions may comprise the formulation of auristatin-antibody conjugates disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722. Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals. Microencapsulation of recombinant proteins for sustained release has been performed successfully with human growth hormone (rhGH), interferon-(rhIFN−), interleukin-2, and MN rgp120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids may be cleared quickly within the human body. Moreover, the degradability of this polymer may be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,481A, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha et al., J. Control. Release 90, 261 [2003], Zhu et al., Nat. Biotechnol. 18, 24 [2000], and Dai et al., Colloids Surf B Biointerfaces 41, 117 [2005].

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141).

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which an auristatin-antibody conjugate disclosed herein has been absorbed or encapsulated. Where an implantation device is used, the device may be implanted into any suitable tissue or organ, and delivery of an auristatin-antibody conjugate disclosed herein may be directly through the device via bolus, or via continuous administration, or via catheter using continuous infusion.

A pharmaceutical composition comprising an auristatin-antibody conjugate disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations containing an auristatin-antibody conjugate disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Another preparation may involve an effective quantity of an auristatin-antibody conjugate disclosed herein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

### Methods of Producing Auristatin-Antibody Conjugates

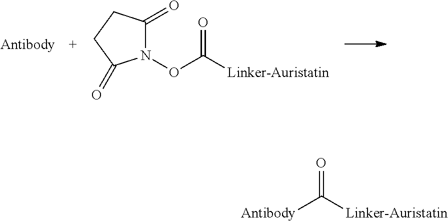

In one aspect, an auristatin-antibody conjugate disclosed herein is prepared through the coupling of an antibody with an auristatin comprising a PEG linker with a N-hydroxy-succinimide (NHS) ester at one end. The auristatin may be any auristatin described herein, including but not limited to, auristatin D, auristatin E, auristatin F, monomethyl auristatin D, monomethyl auristatin E, and monomethyl auristatin F. The NHS containing PEG linker may comprise one or more amino acids. The amino acid(s) may be selected from any natural or unnatural amino acid. The amino acid may be valine. The amino acid may be citrulline. The amino acids may be valine and citrulline. In the NHS containing PEG linkers comprising amino acids, the PEG may be attached to the N or C terminus of the amino acid. In addition, the NHS containing PEG linker may be attached to the auristatin through its phenyl group or by an amino group on the auristatin.

### Therapeutic Use

The auristatin-antibody conjugates may be used in the treatment of a disease or condition in a subject in need thereof. The auristatin-antibody conjugates disclosed herein may be used in the manufacture of a medicament for the treatment of a disease. The methods of treating a disease or condition in a subject in need thereof may comprise administering any of the auristatin-antibody conjugates disclosed herein to the subject. The auristatin-antibody conjugates disclosed herein may be used to treat one or more diseases or conditions in a subject in need thereof. The disease or condition may be a cell proliferative disorder. The disease or condition may be a cancer. The disease or condition may be an autoimmune disease.

The cancer may be a recurrent and/or refractory cancer. The cancer may be an acute cancer. The cancer may be a chronic cancer. The cancer may be a recurrent cancer. The cancer may be a refractory cancer accelerated. The cancer may be in remission. The cancer may be a stage I, stage II, stage III, or stage IV cancer. The cancer may be a juvenile cancer. The cancer may be an adult cancer. Examples of cancers include, but are not limited to, sarcomas, carcinomas, lymphomas or leukemias. The disease or condition may be a cell proliferative disorder. The cell proliferative disorder may be selected from a solid tumor, a lymphoma, a leukemia and a liposarcoma. The cancer may be selected from myelogenous leukemia, lymphoblastic leukemia, myeloid leukemia, an acute myeloid leukemia, myelomonocytic leukemia, neutrophilic leukemia, myelodysplastic syndrome, B-cell lymphoma, burkitt lymphoma, large cell lymphoma, mixed cell lymphoma, follicular lymphoma, mantle cell lymphoma, hodgkin lymphoma, recurrent small lymphocytic lymphoma, hairy cell leukemia, multiple myeloma, basophilic leukemia, eosinophilic leukemia, megakaryoblastic leukemia, monoblastic leukemia, monocytic leukemia, erythroleukemia, erythroid leukemia and hepatocellular carcinoma. The cancer may comprise a hematological malignancy. The hematological malignancy may comprise a B cell malignancy. The cancer may comprise a chronic lymphocytic leukemia. The cancer may comprise an acute lymphoblastic leukemia. The cancer may comprise a CD19-positive Burkitt's lymphoma.

The cancer may be a neuroendocrine cancer. The cancer may comprise a pancreatic cancer. The cancer may comprise an exocrine pancreatic cancer. The cancer may comprise a thyroid cancer. The thyroid cancer may comprise a medullary thyroid cancer.

The cancer may comprise a prostate cancer. The prostate cancer may be a PSMA-positive prostate cancer. PSMA expression may be highly upregulated and restricted to cancer cells in some or all stages of the prostate cancer. The cancer may be hormone-refractory prostate cancer.

The cancer may comprise an epithelial cancer. The cancer may comprise a breast cancer. The cancer may comprise an endometrial cancer. The cancer may comprise an ovarian cancer. The ovarian cancer may comprise a stromal ovarian cancer. The cancer may comprise a cervical cancer.

The cancer may comprise a skin cancer. The skin cancer may comprise a neo-angiogenic skin cancer. The skin cancer may comprise a melanoma.

The cancer may comprise a kidney cancer.

The cancer may comprise a lung cancer. The lung cancer may comprise a small cell lung cancer. The lung cancer may comprise a non-small cell lung cancer.

The cancer may comprise a colorectal cancer. The cancer may comprise a gastric cancer. The cancer may comprise a colon cancer.

The cancer may comprise a brain cancer. The brain cancer may comprise a brain tumor. The cancer may comprise a glioblastoma. The cancer may comprise an astrocytoma.

The cancer may comprise a blood cancer. The blood cancer may comprise a leukemia. The leukemia may comprise a myeloid leukemia. The cancer may comprise a lymphoma. The lymphoma may comprise a non-Hodgkin's lymphoma.

The cancer may comprise a sarcoma. Generally, sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma). The sarcoma may comprise an Ewing's sarcoma.

The cancer may be a carcinoma. Generally, carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis.

In some instances, the cancer is a lung cancer. Lung cancer may start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

The cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

The cancer may be a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

The cancer may comprise a solid tumor. The cancer may comprise a sarcoma. The cancer may be selected from a group consisting of a bladder cancer, a breast cancer, a colon cancer, a rectal cancer, an endometrial cancer, a kidney cancer, a lung cancer, melanoma, a myeloma, a thyroid cancer, a pancreatic cancer, a glioma, a malignant glioma of the brain, a glioblastoma, an ovarian cancer, a prostate cancer and a PSMA-positive prostate cancer. The cancer may have non-uniform antigen expression. The cancer may have modulated antigen expression. The antigen may be a surface antigen. The cancer may not comprise a myeloma. The cancer may not comprise a melanoma. The cancer may not comprise a colon cancer. The cancer may be acute lymphoblastic leukemia (ALL). The cancer may be relapsed ALL. The cancer may be refractory ALL. The cancer may be relapsed, refractory ALL. The cancer may be chronic lymphocytic leukemia (CLL). The cancer may be relapsed CLL. The cancer may be refractory CLL. The cancer may be relapsed, refractory CLL.

The cancer may be a breast cancer. The breast cancer may be triple positive breast cancer (estrogen receptor, progesterone receptor and Her2 positive). The breast cancer may be triple negative breast cancer (estrogen receptor, progesterone receptor and Her2 negative). The breast cancer may be estrogen receptor positive. The breast cancer may be estrogen receptor negative. The breast cancer may be progesterone receptor positive. The breast cancer may be progesterone receptor negative. The breast cancer may comprise a Her2 negative breast cancer. The breast cancer may comprise a low-expressing Her2 breast cancer. The breast cancer may comprise a Her2 positive breast cancer. Cell lines expressing Her2 have been well-characterized for antigen density, reflecting clinical immunohistochemistry characterization which classifies malignancies as 0 (<20,000 Her2 antigens per cell), 1+(100,000 Her2 antigens per cell), 2+(500,000 Her2 antigens per cell), and 3+(>2,000,000 Her2 antigens per cell). The present invention provides for methods of treating breast cancers of these classifications. The breast cancer may comprise a breast cancer classified as Her2 0. The breast cancer may comprise a breast cancer classified as Her2 1+. The breast cancer may comprise a breast cancer classified as Her2 2+. The breast cancer may comprise a breast cancer classified as a Her2 3+.

The disease or condition may be an autoimmune disease or an autoimmune disease related condition. The autoimmune disease and/or autoimmune disease related condition may include, but is not limited to, acute disseminated encephalomyelitis, alopecia areata, antiphospholipid syndrome, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendrocrine syndrome, autoimmune progesterone dermatitis, autoimmune thrombocytopenic purpura, autoimmune urticaria, autoimmune uveitis, Behcet's disease, Celiac disease, cold agglutinin disease, Crohn's disease, dermatomyositis, diabetes mellitus type 1, eosinophilic fasciitis, gastrointestinal pemphigoid, Goodpasture's syndrome, Grave's disease, Guillain-Barre syndrome, Hashimoto's encephalopathy, Hashimoto's thyroiditis, idiopathic thrombocytopenic purpura, lupus erythematosus, Miller-Fisher syndrome, mixed connective tissue disease, multiple sclerosis, myasthenia gravis, narcolepsy, pemphigus vulgaris, pernicious anaemia, polymyositis, primary biliary cirrhosis, psoriasis, psoriatic arthritis, relapsing polychondritis, rheumatoid arthritis, rheumatic fever, Sjogren's syndrome, temporal arteritis, transverse myelitis, ulcerative colitis, undifferentiated connective tissue disease, vasculitis, and Wegener's granulomatosis.

The autoimmune disease may be a T-cell mediated autoimmune disease. T-cell mediated autoimmune diseases include, but are not limited to, multiple sclerosis, type-1 diabetes, and psoriasis. In other instances, the autoimmune disease lupus, Sjogren's syndrome, scleroderma, rheumatoid arthritis, dermatomyositis, Hasmimoto's thyroiditis, Addison's disease, celiac disease, Crohn's disease, pernicious anemia, pemphigus vulgaris, vitiligo, autoimmune hemolytic anemia, idiopathic thrombocytopenic purpura, myasthenia gravis, Ord's thyroiditis, Graves' disease, Guillain-Barre syndrome, acute disseminated encephalomyelitis, opsoclonus-myoclonus syndrome, ankylosing spondylitisis, antiphospholipid immunoglobulin syndrome, aplastic anemia, autoimmune hepatitis, Goodpasture's syndrome, Reiter's syndrome, Takayasu's arteritis, temporal arteritis, Wegener's granulomatosis, alopecia universalis, Behcet's disease, chronic fatigue, dysautonomia, endometriosis, interstitial cystitis, neuromyotonia, scleroderma, and vulvodynia. Lupus can include, but may be not limited to, acute cutaneous lupus erythematosus, subacute cutaneous lupus erythematosus, chronic cutaneous lupus erythematosus, discoid lupus erythematosus, childhood discoid lupus erythematosus, generalized discoid lupus erythematosus, localized discoid lupus erythematosus, chilblain lupus erythematosus (hutchinson), lupus erythematosus-lichen planus overlap syndrome, lupus erythematosus panniculitis (lupus erythematosus profundus), tumid lupus erythematosus, verrucous lupus erythematosus (hypertrophic lupus erythematosus), complement deficiency syndromes, drug-induced lupus erythematosus, neonatal lupus erythematosus, and systemic lupus erythematosus. The autoimmune disease or condition may be multiple sclerosis. The autoimmune disease or condition may be diabetes.

### EXAMPLES

The following illustrative examples are representative of embodiments of the compositions and methods described herein and are not meant to be limiting in any way. All cell lines were purchased from ATCC unless otherwise stated. Ramos, Jurkat, OPM-2 cells were cultured in RPMI with 10% FBS and 100 U/mL Penicillin Streptomycin. MDA-MB-231 cells were cultured in DMEM (high glucose) with 10% FBS, 1 mM sodium pyruvate, and 100 U/mL Penicillin Streptomycin. SKBR-3 cells were cultured in DMEM with 10% FBS and 1x Antibiotic-Antimycotic (100 U/mL Penicillin Streptomycin, 0.25 μg/mL Fungizone). CHO—S cells (Life Technologies) were cultured in FreeStyle CHO Expression Medium (Life Technologies) supplemented with 8 mM glutamine.

### Example 1

### Cytotoxicity of Auristatins and Anti-CXCR4-Auristatin Conjugates

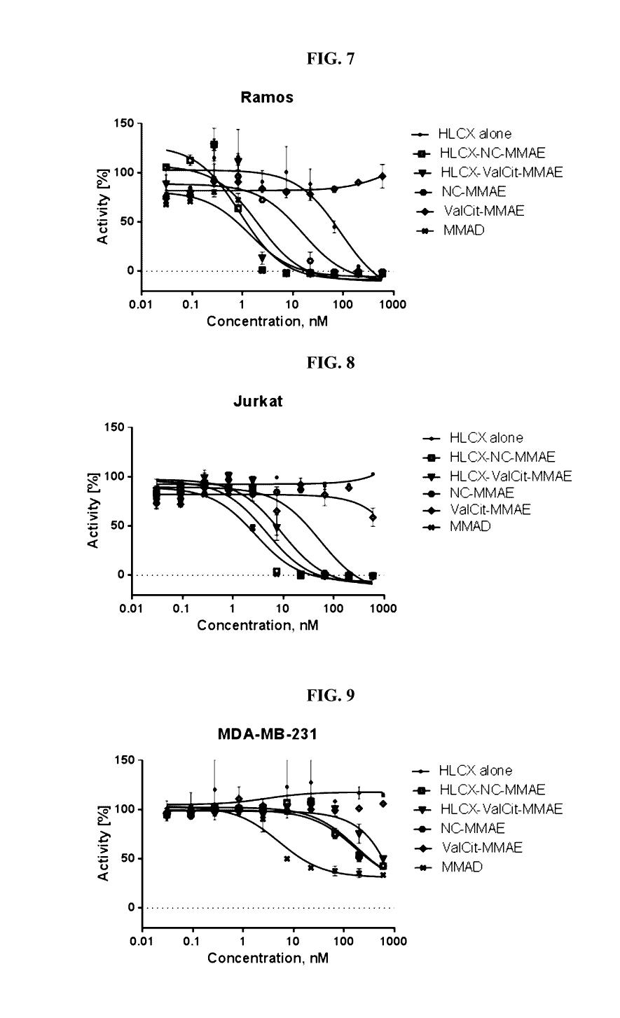

Ramos (CXCR4+), Jurkat (CXCR4+), and MDA-MB-231 (CXCR4−) cells were treated with either small molecule drugs, anti-CXCR4 (SEQ ID NOS: 3, 4), or anti-CXCR4-auristatin conjugates with concentrations ranging from 600 nM to 30 pM for 4 days at 37° C. Cell viabilities were measured by addition of CellTiter-Glo (Promega) and the luminescent signals were read by EnVision plate reader. IC50s are shown in Table 1, which indicates anti-CXCR4-NC-MMAE and anti-CXCR4-ValCit-MMAE conjugates are highly potent in CXCR4+ cells (Ramos, Jurkat), relative to the antibody alone or drug alone (NC-MMAE, ValCit-MMAE). FIGS. 7-9 show the activity curves corresponding to the IC50 values shown in Table 1.

TABLE 1
Cytotoxicity of auristatin and anti-CXCR4-auristatin
conjugates towards different cell lines.

| | IC₅₀ (nM) | | |
|---|---|---|---|
| ADC/SM | Ramos | Jurkat | MDA-MB-231 |
| ANTI-CXCR4 | 89.4 | — | — |
| ANTI-CXCR4-NC- | 0.89 | 4.6 | 178 |
| MMAE | | | |
| ANTI-CXCR4-ValCit- | 2 | 9.2 | >1000 |
| MMAE | | | |
| NC-MMAE | 15.5 | 53 | 167 |
| ValCit-MMAE | 929 | >1000 | — |
| MMAD | 1.4 | 2.8 | 5.2 |

Briefly, 1000 cells/well were seeded in a 384-well plate (45 uL/well with their corresponding media) and incubated at 37° C. overnight. On the day of the drug treatment, serial dilution of either the small molecule drugs, antibody alone, or anti-CXCR4-auristatin conjugates were prepared starting at 6 μM as top concentration followed by a 3-fold dilution in PBS. 5 μL (10×) of each of these dilutions were then added into the wells containing the cells (final volume was 50 μL, final top concentration of the drug was 600 nM). Control wells without the drugs (containing DMSO in PBS only) were also prepared along with wells treated with Staurosporine (as positive control). Plates were covered with metal lids and incubated at 37° C. for 4 days. CellTiter-Glo (Promega) were first diluted 1:2 (CellTiter-Glo: H₂O) before addition of 30 μL into each well using Kalypsys bottle valve. Plates were shaken and incubated for at least 10 min before the generated luminescent signals were read using PerkinElmer EnVision Multilabel Reader.

### Example 2

### NHS-ValCit-MMAE Synthesis

Step 1. Nitration and Boc Protection.

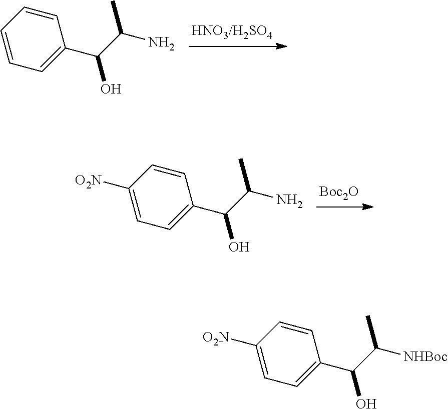 [SMILES: "Boc2O" ; C[C@@H](N)[C@@H](O)c1ccccc1 ; C[C@@H](N)[C@@H](O)c1ccc([N+](=O)[O-])cc1 ; C[C@@H](NC(=O)OC(C)(C)C)[C@@H](O)c1ccc([N+](=O)[O-])cc1]

Concentrated H₂SO₄ (1 mL) was slowly added to the aminoalcohol (300 mg, 2 mmol) at 0° C. followed by conc. HNO₃ (1 mL). The mixture was stirred at 0° C. for 1 h and then poured on to ice. The mixture was neutralized with 4 N KOH to pH 11 and extracted with ethyl acetate. The combined organic layers were dried with sodium sulfate and concentrated to give a residue, which was dissolved in MeOH (5 mL). Boc₂O (523 mg, 1.2 equiv) was added, and the mixture was stirred at room temp overnight. The reaction mixture was concentrated and the residue was purified by chromatography on silica gel (50% EA/hexane) to give the product as an oil (180 mg, 30%), MS: 297 (M+H). ¹H-NMR (CDCl₃) δ 8.22 (d, 2H), 7.53 (d, 2H), 4.75 (m, 1H), 4.7 (brs, 1H), 3.82 (m, 1H), 3.7 (m, 1H), 1.38 (s, 9H), 1.35 (d, 3H).

Step 2. Reduction of Nitro Group and Couple with Fmoc-Val-Cit-OH.

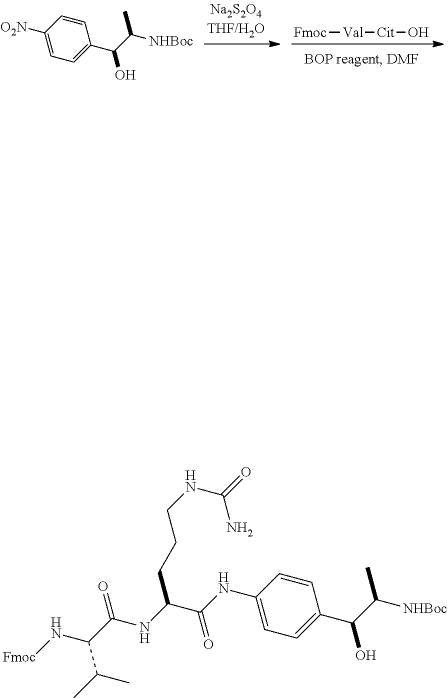

To a solution of the above nitro amino alcohol (300 mg, 1 mmol) in THF/H₂O (10 mL, 1/1) was added Na₂S₂O₄ (500 mg). The reaction mixture was stirred at room temp for 2 h and partitioned between ethylacetate and water. The combined organic layers was dried over sodium sulfate and concentrated to give the aniline (212 mg) which was used for next reaction directly.

A mixture of the above aniline, Fmoc-val-Cit-OH (791 mg, 1.6 mmol) and BOP reagent (708 mg, 1.6 mmol) in DMF (3 mL) was stirred overnight. Reverse-phase prep-HPLC of the residue gave the compound as a white solid (180 mg, 24% for two steps), MS: 745 (M+H). HPLC retention time: 8.7 min (Agilent Eclipse XDB-C18, 5 um, 4.6×150 mm column 10-70% CH3CN/H2O, 16 min, 220 nm).

Step 3. Boc Deprotection, Coupling with Val-Val-Dil-Dap and Fmoc Deprotection.

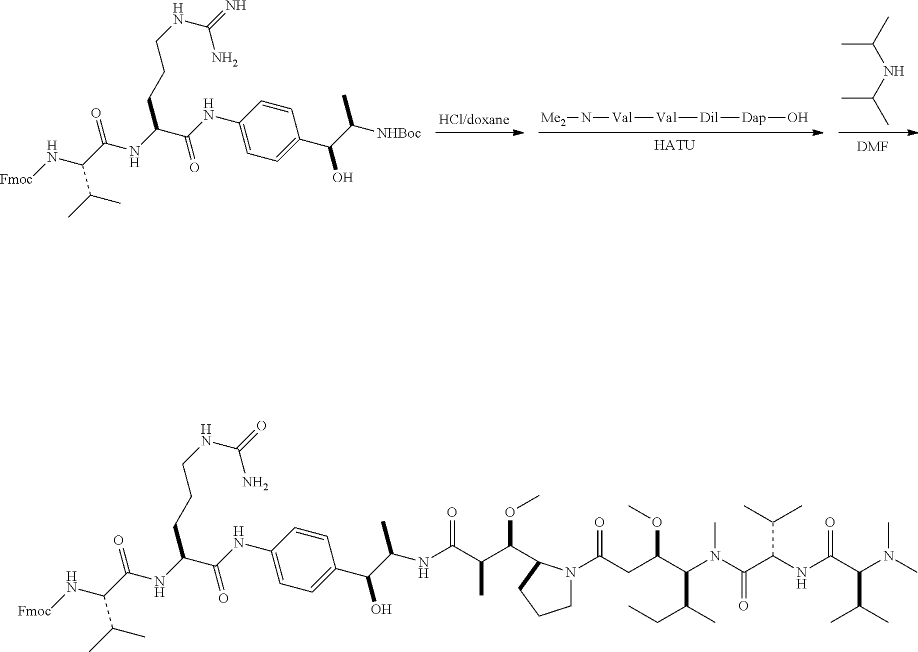

The above peptide (180 mg, 0.242 mmol) was dissolved in 4N HCl (2 mL). After stirring for 4 h, the mixture was concentrated and DMF (2 mL) was added, followed by Di-Me-N-Val-Val-Dil-Dap-OH (160 mg, 0.266 mmol) and diisopropyl ethyl amine (0.13 mL). Then HATU (110 mg, 0.29 mmol) was added. After 4 h, diisopropylamine (2 mL) was added and the mixture was stirred overnight. The mixture was concentrated. Prep HPLC (10-70% ACN/H2O) of the residue gave the compound as a white solid (64 mg, 26% for three steps). MS: 1003 (M+H). HPLC retention time: 8.5 min (Agilent Eclipse XDB-C18, 5 um, 4.6×150 mm column 10-60% CH3CN/H2O, 16 min, 220 nm).

Step 4. Coupling with PEG4 Diacid and N-Hydroxysuccinimide (NETS).

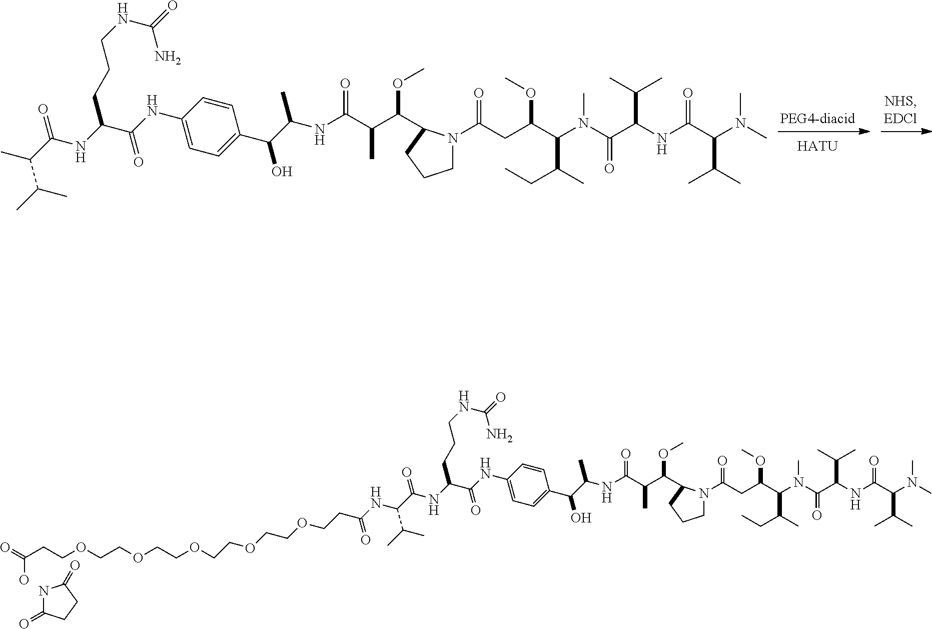

A mixture of amine (64 mg, 0.064 mmol), PEG4-diacid (86 mg, 0.256 mmol), HATU (24 mg, 0.68 mmol) and diisopropyl ethyl amine (0.14 mL) in DMF (1 mL) was stirred at rt for 2 h. The mixture was purified by prep-HPLC (5-40% ACN/H2O) to give the product as a white solid (23 mg). HPLC retention time: 7.8 min (Agilent Eclipse XDB-C18, 5 um, 4.6×150 mm column 10-60% CH3CN/H2O, 16 min, 220 nm).

A mixture of the above white solid (23 mg), N-hydroxysuccinimide (6 mg, 0.052 mmol) and EDCI (10 mg, 0.052 mmol) in DMF/DCM (20%, 1 ml) was stirred at room temperature for 2 h. The mixture was concentrated and purified by prep HPLC (5-40% ACN/H2O) to give the final product as a white solid (23 mg, 25% for two steps). MS: 1420 (M+H). HPLC retention time: 8.02 min (Agilent Eclipse XDB-C18, 5 um, 4.6×150 mm column 10-60% CH3CN/H2O, 16 min, 220 nm).

### Example 3

### NHS—NC-MMAE Synthesis

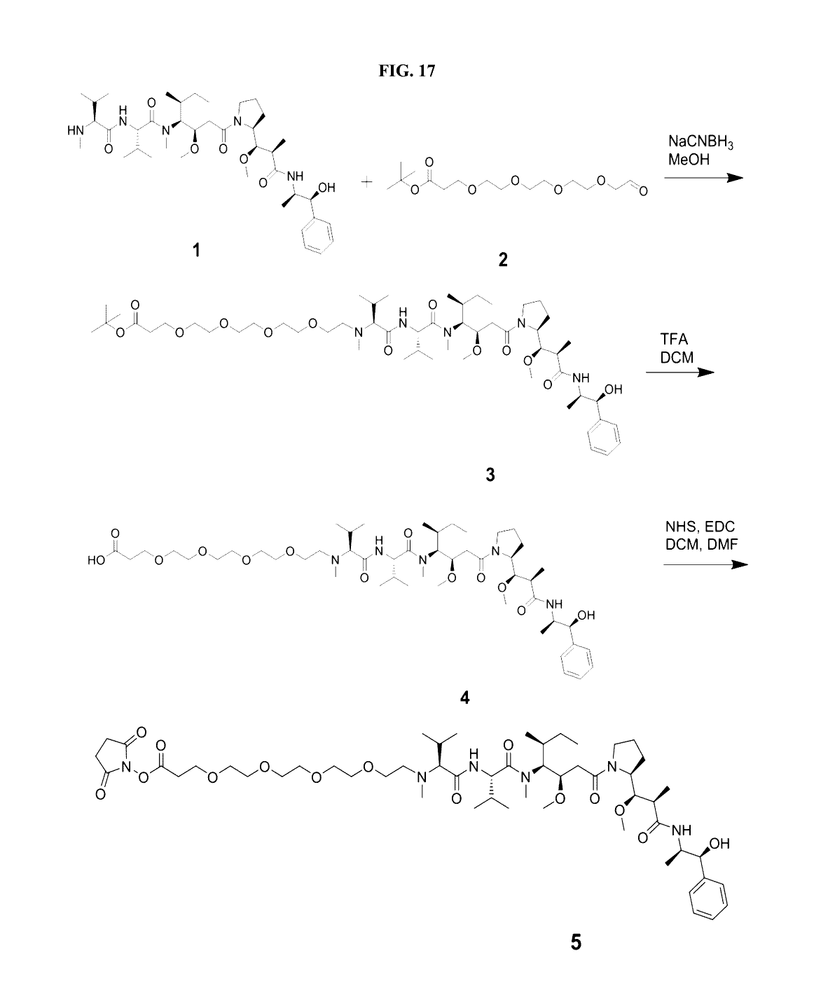

FIG. 17 depicts a synthesis scheme for NHS—NC-MMAE. Compounds in the figure are numbered and referred to in this example.

Preparation of Compound 3:

To a solution of MMAE (1) (100 mg, 0.12 mmol) in 2 mL of MeOH was added aldehyde 2 (120 mg, 0.37 mmol) and NaCNBH₃ (19 mg, 0.31 mmol). After 1 h, reaction was done by LCMS, and the mixture was purified by HPLC (ACN/H₂O/TFA) to give compound 3 (128 mg, 94%) as TFA salt. LCMS (Column: Phenomenex Gemini NX, 5u, C18, 50×4.6 mm, ACN/H2O (0.1% HCOOH) 5-95%, 5 min) retention time 1.83 min. MS m/z 1022.4 (M+H).

Preparation of Compound 4:

To a solution of compound 3 (128 mg, 0.11 mmol) in 4 mL of DCM was added 5 mL of TFA. After 10 min, reaction was done by LCMS, and the mixture was diluted with 20 mL of ACN, evaporated, and purified by HPLC (ACN/H₂O/TFA) to give compound 4 (85 mg, 80%). LCMS (Column: Phenomenex Gemini NX, 5u, C18, 50×4.6 mm, ACN/H2O (0.1% HCOOH) 5-95%, 5 min) retention time 1.68 min. MS m/z 966.6 (M+H).

Preparation of Compound 5:

To a solution of compound 4 (85 mg, 0.088 mmol) in 3 mL of DCM and 2 mL of DMF was added NHS (28 mg, 0.24 mmol) and EDC (45 mg, 0.24 mmol). After 30 min, reaction was done by LCMS. DCM was evaporated, and the mixture was purified by HPLC to give compound 5 (72 mg, 77%). LCMS (Column: Phenomenex Gemini NX, 5u, C18, 50×4.6 mm, ACN/H2O (0.1% HCOOH) 5-95%, 5 min) retention time 1.71 min. MS m/z 1063.8 (M+H).

### Example 4

### Auristatin E Amine Synthesis

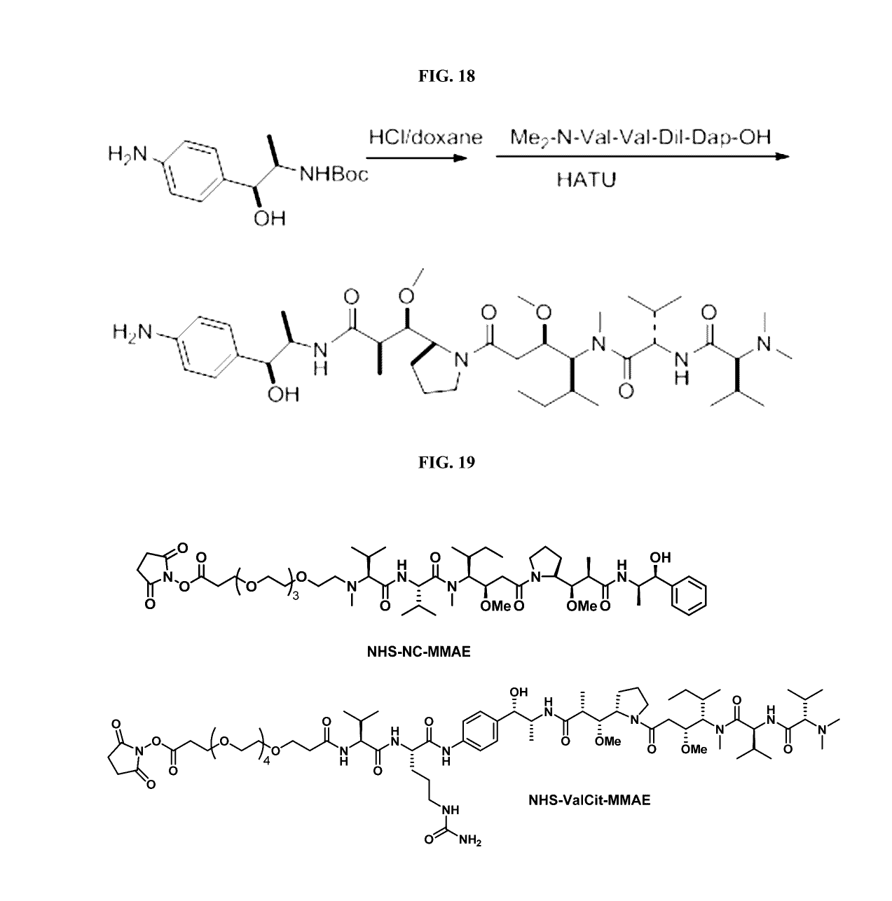

Auristatin E amine is used in cell killing assays described herein. Synthesis of auristatin E amine is depicted in FIG. 18.

Procedure: The anilide (22 mg, 0.082 mmol) was dissolved in 4N HCl (2 mL). After stirring for 4 h, the mixture was concentrated and DMF (2 mL) was added, followed by Di-Me-N-Val-Val-Dil-Dap-OH.TFA (58 mg, 0.082 mmol) and diisopropyl ethyl amine (0.044 mL). Then HATU (31 mg, 0.082 mmol) was added. After 30 min, the mixture was subject to preparative HPLC (2-40% ACN/H₂O) to give the desired compound as a white solid (11 mg). MS: 747 (M+H).

Analytical HPLC Conditions:

Column: HPLC Gemini-NX-5 u, C18, 4.6×150 mm

Conditions: CH3CN—H2O 10-60%, 16 min, UV 220 nm

Purity observed: 99.7%

### Example 5

### Expression and Purification of Anti-CXCR4 IgG Antibody

Anti-CXCR4 IgG antibody was expressed in suspension HEK 293F cells (Life Technologies) by transient gene transfection of two plasmids, namely pFUSE-HLCX HC and pFUSE-HLCX LC (see SEQ ID NOS: 1 and 2) in 2:1 (heavy chain: light chain) ratio. One day before transfection, cells were seeded at a density of 0.5×10⁶ cells/mL (150 mL) in a 500-mL flat-bottomed shake flask using FreeStyle 293 Expression Medium (Life Technologies) and was incubated at 37° C. (with 125 rpm shaking). Upon reaching a density of 1.0×10⁶ cells/mL, cells were spun down and resuspended carefully in fresh prewarmed media. Transfection was done using 293 fectin following manufacturer's protocol. Harvest was done every 48 h for a total of three harvests (6-day expression). Briefly, cells were spun down and the supernatant was collected and filtered (0.22 μm). To prevent the degradation of antibody, protease tab inhibitor (Roche) was also added to the supernatant and was kept on ice at all times. The cell pellet was resuspended carefully in 150 mL of prewarmed media and returned to the incubator/shaker to be harvested again after 48 h. On the other hand, the collected supernatant was immediately purified by passing through a Protein G column (1-mL resin) previously equilibrated with 1x PBS. The flow thru was passed through the column again before washing with 1x PBS, and finally eluted the antibody with 15 mL of 100 mM glycine (pH 2.8), which was later neutralized with 10% volume of 1 M Tris-HCl (pH 8.0). The antibody was then buffer exchanged and concentrated into 1x PBS (Amicon, 30 kDA MWCO by EMD Millipore) giving an overall 3 harvests average yield of 7.7 mg/L.

### Example 6

### Synthesis of Anti-CXCR4 IgG Conjugates

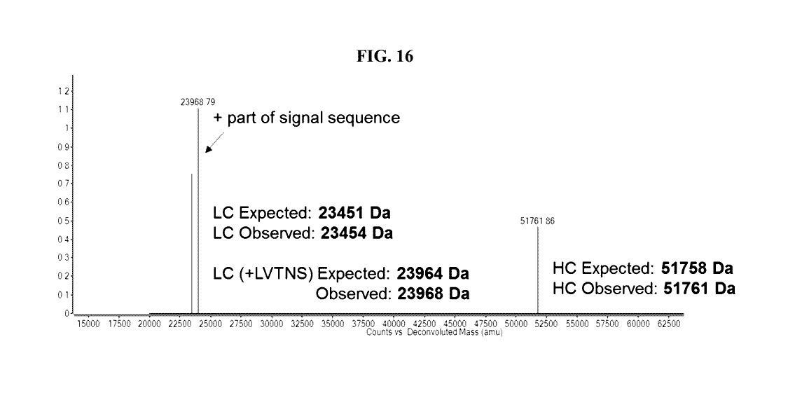

A solution of anti-CXCR4 IgG antibody, SEQ ID NOS: 3, 4 (1.0 mg, final concentration of antibody was 2 mg/mL) in PBS, pH 7.4 was reacted with 80 equiv of the NHS—NC-MMAE or NHS-ValCit-MMAE (NHS—NC-MMAE and NHS-ValCit-MMAE are depicted in FIG. 19) with addition of up to 10% DMSO. The mixture was left to react at room temperature for 16-20 h. Afterwards, the resulting ADCs were isolated from excess small molecule using the Amicon filter (30 kDa MWCO by EMD Millipore) giving rise to 69% yield for anti-CXCR4-NC-MMAE and 78% yield for anti-CXCR4-ValCit-MMAE. The identities of the resulting ADCs were confirmed by SDS-PAGE and ESI-MS analyses (see FIGS. 4 and 16). As for the synthesis of the anti-CXCR4 IgG-AF488, 2-5 mg/mL of the antibody in PBS (pH 7.4) was reacted with 30-50 equiv of the Alexa Fluor 488 carboxylic acid, succinimidyl ester (Life Technologies) overnight at room temperature. Afterwards, the dye-conjugated antibody was isolated from excess small molecule using Amicon filter (30 kDa MWCO by EMD Millipore).

### Example 7

### Binding Assay

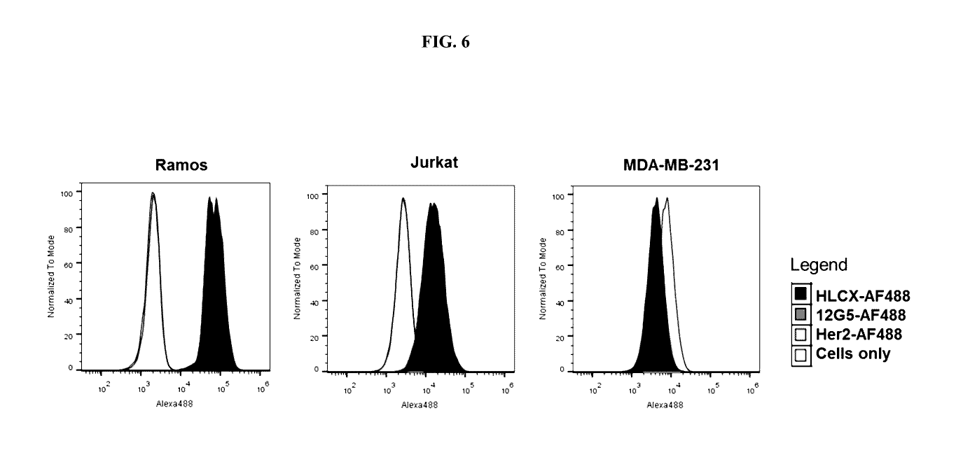

An appropriate number of cells (500 k/well) were plated in a 96-well plate. Cells were treated with 100 μL of 50 nM each of the Alexa Fluor 488-antibody conjugates (except for the control cells) in FACS buffer (2% BSA, 1 mM EDTA, in 1x PBS) at 4° C. in the dark, with gentle rocking. After 1 h, cells were washed 3x (200 μL of cold FACS buffer, with repeated centrifugation and cell resuspension). Finally, cells were resuspended in 150 μL of the same buffer for subsequent FACS analysis (BD Accuri). All the results were processed using FlowJo software. FIG. 6 shows the FACS analysis of anti-CXCR4-AF488 binding to the CXCR4+ Ramos and Jurkat cells.

TABLE 2
Antibody Sequences

| | SEQ | |
|---|---|---|
| Description | ID NO: | Sequence |
| HLCX | 1 | CAGGTGCAGCTGGTGGAGTCTGGAG |
| heavy chain | | GAGGCTTGGTCCAGCCTGGGGGGTC |
| coding | | CCTGAGACTCTCCTGTGCAGCCTCT |
| sequence | | GGGTTCAATATTAAGGACACTTACA |
| | | TCCACTGGGTCCGCCAGGCTCCAGG |
| | | GAAGGGGCTGGAGTGGGTCGCACGT |
| | | ATTTATCCTACCAATGGTTACACAC |
| | | GCTACGCAGACTCCGTGAAGGGCCG |
| | | ATTCACCATCTCCGCAGACACTTCC |
| | | AAGAACACGGCGTATCTTCAAATGA |
| | | ACAGCCTGAGAGCCGAGGACACGGC |
| | | CGTGTATTACTGTTCGAGAGAAACT |
| | | AAGAAATACCAGAGCTATCGCAAAT |
| | | GTAGAGGAGGCCGAAGGTGGTGCTA |
| | | CCAAAAGTCTTATACCTACAATTAT |
| | | GAAGACTACTGGGGCCAAGGAACCC |
| | | TGGTCACCGTCTCCTCAGCCTCCAC |
| | | CAAGGGCCCATCGGTCTTCCCCCTG |
| | | GCACCCTCCTCCAAGAGCACCTCTG |
| | | GGGGCACAGCGGCCCTGGGCTGCCT |
| | | GGTCAAGGACTACTTCCCCGAACCG |
| | | GTGACGGTGTCGTGGAACTCAGGCG |
| | | CCCTGACCAGCGGCGTGCACACCTT |
| | | CCCGGCTGTCCTACAGTCCTCAGGA |
| | | CTCTACTCCCTCAGCAGCGTGGTGA |
| | | CTGTGCCCTCTAGCAGCTTGGGCAC |
| | | CCAGACCTACATCTGCAACGTGAAT |
| | | CACAAGCCCAGCAACACCAAGGTGG |
| | | ACAAGAAAGTTGAACCCAAATCTTG |
| | | CGACAAAACTCACACATGCCCACCG |
| | | TGCCCAGCACCTGAACTCCTGGGGG |
| | | GACCGTCAGTCTTCCTCTTCCCCCC |
| | | AAAACCCAAGGACACCCTCATGATC |
| | | TCCCGGACCCCTGAGGTCACATGCG |
| | | TGGTGGTGGACGTGAGCCACGAAGA |
| | | CCCTGAGGTCAAGTTCAACTGGTAC |
| | | GTGGACGGCGTGGAGGTGCATAATG |
| | | CCAAGACAAAGCCGCGGGAGGAGCA |
| | | GTACAACAGCACGTACCGTGTGGTC |
| | | AGCGTCCTCACCGTCCTGCACCAGG |
| | | ACTGGCTGAATGGCAAGGAGTACAA |
| | | GTGCAAGGTCTCCAACAAAGCCCTC |
| | | CCAGCCCCCATCGAGAAAACCATCT |
| | | CCAAAGCCAAAGGGCAGCCCCGAGA |
| | | ACCACAGGTGTACACCCTGCCCCCA |
| | | TCCCGGGATGAGCTGACCAAGAACC |
| | | AGGTCAGCCTGACCTGCCTGGTCAA |
| | | AGGCTTCTATCCCAGCGACATCGCC |
| | | GTGGAGTGGGAGAGCAATGGGCAGC |
| | | CGGAGAACAACTACAAGACCACGCC |
| | | TCCCGTGCTGGACTCCGACGGCTCC |
| | | TTCTTCCTCTACAGCAAGCTCACCG |
| | | TGGACAAGAGCAGGTGGCAGCAGGG |
| | | GAACGTCTTCTCATGCTCCGTGATG |
| | | CATGAGGCTCTGCACAACCACTACA |
| | | CGCAGAAGAGCCTCTCCCTGTCTCC |
| | | GGGTAAA |
| HLCX | 2 | GACATCCAGATGACCCAGTCTCCAT |
| light chain | | CCTCCCTGTCTGCATCTGTAGGAGA |
| coding | | CAGAGTCACCATCACTTGCCGGGCA |
| sequence | | AGTCAGGATGTGAATACCGCGGTCG |
| | | CATGGTATCAGCAGAAACCAGGGAA |
| | | AGCCCCTAAGCTCCTGATCTATTCT |
| | | GCATCCTTCTTGTATAGTGGGGTCC |
| | | CATCAAGGTTCAGTGGCAGTAGATC |
| | | TGGGACAGATTTCACTCTCACCATC |
| | | AGCAGTCTGCAACCTGAAGATTTTG |
| | | CAACTTACTACTGTCAACAGCATTA |
| | | CACTACCCCTCCGACGTTCGGCCAA |
| | | GGTACCAAGCTTGAGATCAAACGAA |
| | | CTGTGGCTGCACCATCTGTCTTCAT |
| | | CTTCCCGCCATCTGATGAGCAGTTG |
| | | AAATCTGGAACTGCCTCTGTCGTGT |
| | | GCCTGCTGAATAACTTCTATCCCAG |
| | | AGAGGCCAAAGTACAGTGGAAGGTG |
| | | GATAACGCCCTCCAATCGGGTAACT |
| | | CCCAGGAGAGTGTCACAGAGCAGGA |
| | | CAGCAAGGACAGCACCTACAGCCTC |
| | | AGCAGCACCCTGACGCTGAGCAAAG |
| | | CAGACTACGAGAAACACAAAGTCTA |
| | | CGCCTGCGAAGTCACCCATCAGGGC |
| | | CTGTCCTCGCCCGTCACAAAGAGCT |
| | | TCAACAGGGGAGAGTGT |
| HLCX | 3 | QVQLVESGGGLVQPGGSLRLSCAAS |
| heavy chain | | GFNIKDTYIHWVRQAPGKGLEWVAR |
| protein | | IYPTNGYTRYADSVKGRFTISADTS |
| sequence | | KNTAYLQMNSLRAEDTAVYYCSRET |
| | | KKYQSYRKCRGGRRWCYQKSYTYNY |
| | | EDYWGQGTLVTVSSASTKGPSVFPL |
| | | APSSKSTSGGTAALGCLVKDYFPEP |
| | | VTVSWNSGALTSGVHTFPAVLQSSG |
| | | LYSLSSVVTVPSSSLGTQTYICNVN |
| | | HKPSNTKVDKKVEPKSCDKTHTCPP |
| | | CPAPELLGGPSVFLFPPKPKDTLMI |
| | | SRTPEVTCVVVDVSHEDPEVKFNWY |
| | | VDGVEVHNAKTKPREEQYNSTYRVV |
| | | SVLTVLHQDWLNGKEYKCKVSNKAL |
| | | PAPIEKTISKAKGQPREPQVYTLPP |
| | | SRDELTKNQVSLTCLVKGFYPSDIA |
| | | VEWESNGQPENNYKTTPPVLDSDGS |
| | | FFLYSKLTVDKSRWQQGNVFSCSVM |
| | | HEALHNHYTQKSLSLSPGK |
| HLCX | 4 | DIQMTQSPSSLSASVGDRVTITCRA |
| light chain | | SQDVNTAVAWYQQKPGKAPKLLIYS |
| protein | | ASFLYSGVPSRFSGSRSGTDFTLTI |
| sequence | | SSLQPEDFATYYCQQHYTTPPTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |

What is claimed is:

1. An antibody drug conjugate comprising an auristatin linked to an antibody or fragment thereof through a phenyl group of the auristatin.

2. The antibody drug conjugate of claim 1, wherein the auristatin is of Formula (I): 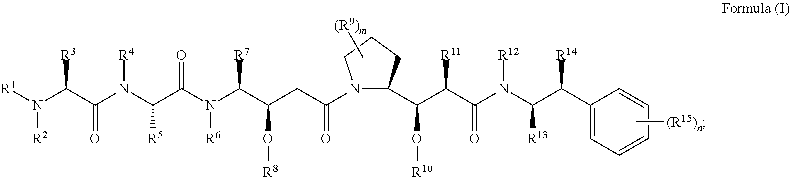
wherein
each R¹, R², R⁴, R⁶, and R¹² is independently hydrogen or optionally substituted alkyl;
each R³, R⁵, R⁷, and R¹¹ is independently optionally substituted alkyl;
each R⁸ and R¹⁰ is independently optionally substituted alkyl;
each R¹³ and R¹⁴ are independently hydrogen, halogen, —CN, —OR^{a}, —COOR^{a}, —N(R^{a})₂, optionally substituted alkyl, optionally substituted C₃-C₈ cycloalkyl, optionally substituted C₂-C₈ heterocycloalkyl, optionally substituted aryl, or optionally substituted 5-, or 6-membered heteroaryl;
each R⁹ is independently hydrogen, halogen, —OR^{a}, —N(R^{a})₂, —SO₂N(R^{a})₂, —CO₂N(R^{a})₂, or optionally substituted alkyl; or 2 R⁹ on the same carbon are taken together to form an oxo; or 2 R⁹ on the adjacent carbons are taken together to form an alkene;
each R¹⁵ is independently hydrogen, halogen, —OR^{a}, —N(R^{a})₂, —SO₂N(R^{a})₂, —CO₂N(R^{a})₂, or optionally substituted alkyl;
each R^{a} is hydrogen or optionally substituted alkyl;
n is 0-5; and
m is 0-3.

3. The antibody drug conjugate of claim 2, wherein:
R² is methyl;
R³ is isopropyl;
R⁴ is hydrogen;
R⁵ is isopropyl;
R⁶ is methyl;
R⁷ is 2-butyl;
R⁸ is methyl;
R⁹ is hydrogen;
R¹⁰ is methyl;
R¹¹ is methyl;
R¹² is hydrogen;
m is 0; and
n is 0.

4. The antibody drug conjugate of claim 3, wherein
R¹ is methyl; R¹³ is methyl; and R¹⁴ is —OH; or
R¹ is hydrogen; R¹³ is methyl; and R¹⁴ is —OH; or
R¹ is methyl; R¹³ is —COOH; and R¹⁴ is hydrogen; or
R¹ is hydrogen; R¹³ is —COOH; and R¹⁴ is hydrogen; or
R¹ is methyl; R¹³ is 2-thiazolyl; and R¹⁴ is hydrogen; or
R¹ is hydrogen; R¹³ is 2-thiazolyl; and R¹⁴ is hydrogen.

5. The antibody drug conjugate of claim 1, wherein the antibody or fragment thereof is linked through a primary amine on a phenyl group of an auristatin amine of Formula (II): 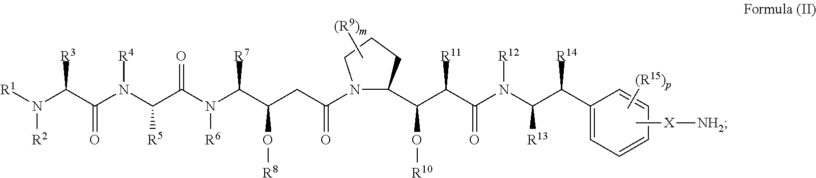
wherein
each R¹, R², R⁴, R⁶, and R¹² is independently hydrogen or optionally substituted alkyl;
each R³, R⁵, R⁷, and R¹¹ is independently optionally substituted alkyl;
each R⁸ and R¹⁰ is independently optionally substituted alkyl;
each R¹³ and R¹⁴ are independently hydrogen, halogen, —CN, —OR^{a}, —COOR^{a}, —N(R^{a})₂, optionally substituted alkyl, optionally substituted C₃-C₈ cycloalkyl, optionally substituted C₂-C₈ heterocycloalkyl, optionally substituted aryl, or optionally substituted 5-, or 6-membered heteroaryl;
each R⁹ is independently hydrogen, halogen, —OR^{a}, —N(R^{a})₂, —SO₂N(R^{a})₂, —CO₂N(R^{a})₂, or optionally substituted alkyl; or 2 R⁹ on the same carbon are taken together to form an oxo; or 2 R⁹ on the adjacent carbons are taken together to form an alkene;
each R¹⁵ is independently hydrogen, halogen, —OR^{a}, —N(R^{a})₂, —SO₂N(R^{a})₂, —CO₂N(R^{a})₂, or optionally substituted alkyl;
each R^{a} is hydrogen or optionally substituted alkyl;
p is 0-4;
m is 0-3; and
X is a bond or C₁-C₆alkylene.

6. The antibody drug conjugate of claim 1, wherein the auristatin amine has the following structure:

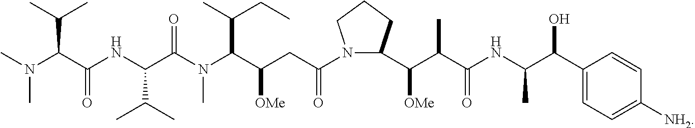 [SMILES: CCC(C)[C@@H]([C@@H](CC(=O)N1CCC[C@H]1[C@H](OC)[C@@H](C)C(=O)N[C@H](C)[C@@H](O)c1ccc(N)cc1)OC)N(C)C(=O)[C@@H](NC(=O)[C@H](C(C)C)N(C)C)C(C)C]

7. The antibody drug conjugate of claim 1, wherein the antibody or fragment thereof binds an antigen on a cancer cell.

8. The antibody drug conjugate of claim 1, wherein the antibody or fragment thereof is an anti-CXCR4 antibody or fragment thereof.

9. The antibody drug conjugate of claim 1, wherein the phenyl group of the auristatin is linked to the antibody or fragment thereof via a linker.

10. The antibody drug conjugate of claim 9, wherein the linker comprises an amino acid.

11. The antibody drug conjugate of claim 10, wherein the amino acid is selected from a valine and a citrulline.

12. The antibody drug conjugate of claim 9, wherein the linker comprises a polyethylene glycol subunit.

13. The antibody drug conjugate of claim 9, wherein the auristatin and the linker together have a structure:

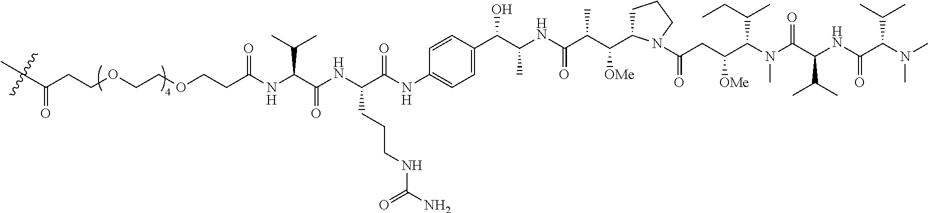

14. A method of treating cancer or an autoimmune disease in a subject in need thereof, the method comprising administering an effective amount of the antibody drug conjugate of claim 1 to the subject.

15. An antibody drug conjugate comprising a monomethyl auristatin linked to an antibody or fragment thereof through a non-cleavable linker.

16. The antibody drug conjugate of claim 15, wherein the non-cleavable linker links the antibody or fragment thereof to a terminal amino group of monomethyl auristatin E.

17. The antibody drug conjugate of claim 15, wherein the monomethyl auristatin E and the non-cleavable linker together have a structure:

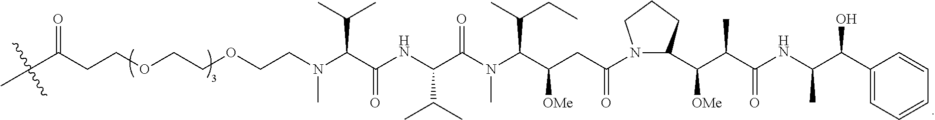

18. A method of treating cancer or an autoimmune disease in a subject in need thereof, the method comprising administering an effective amount of the antibody drug conjugate of claim 15 to the subject.

19. A method of producing an antibody drug conjugate, the method comprising coupling an auristatin with a linker comprising an N-hydroxy-succinimide (NHS) ester.

20. The method of claim 19, wherein coupling the auristatin with the linker comprising the N-hydroxy-succinimide (NHS) ester produces a structure selected from: [chemical structure]


SEQUENCE LISTING (5 sequences)

SEQ ID NO: 1 - DNA, 1407 bp, Artificial Sequence
  Description of Artificial Sequence Synthetic polynucleotide
caggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagagaaactaagaaataccagagctatcgcaaatgtagaggaggccgaaggtggtgctaccaaaagtcttatacctacaattatgaagactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 3 - protein, 469 aa, Artificial Sequence
  Description of Artificial Sequence Synthetic polypeptide
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRETKKYQSYRKCRGGRRWCYQKSYTYNYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Artificial Sequence
  Description of Artificial Sequence Synthetic peptide
LVTNS